(19) United States
(12) Patent Application Publication
(10) Pub. No.: US 2003/0166266 A1
(43) Pub. Date: Sep. 4, 2003

(54) Open reading frame detection compositions and methods

(76) Inventors: Irene Teresa Rombel, Dallas, TX (US); Kathryn F. Sykes, Dallas, TX (US); Stephen Albert Johnston, Dallas, TX (US)

Correspondence Address: Gina N. Shishima, FULBRIGHT & JAWORSKI L.L.P., Suite 2400, 600 Congress Avenue, Austin, TX, 78701, US

(21) Appl. No.: 09/728,041

(22) Filed: Dec. 1, 2000

Related U.S. Application Data
(60) Provisional application No. 60/168,804, filed on Dec. 2, 1999.

(51) Int. Cl. C12N 15/00

(52) U.S. Cl. 435/320.1

(57) ABSTRACT

The present invention relates to a series of plasmid-based expression vectors and methods for systematically screening entire genomes for gene-coding fragments. The compositions and methods described herein facilitate the detection of open reading frames within a DNA sequence. In this manner, the ORF selection vectors of the invention may be utilized in the isolation of genetic vaccine candidates for expression library immunization. The invention allows for the rapid, efficient screening of large genomes of eukaryotic parasites, for example, for determining protective gene-coding DNA fragments

The present application claims the benefit of U.S. Provisional Application Serial No. 60/168,804 filed on Dec. 2, 1999. The entire text of the above-referenced disclosure is herein incorporated by reference.

The government owns rights in the present invention pursuant to DARPA grant number MDA 972-97-1-10013 and grant number 1-R-21-AI-0090-01 from NIH.

### BACKGROUND OF THE INVENTION

1. Field of the Invention

The present invention relates generally to the fields of molecular biology and immunology. More particularly, it concerns methods and compositions involving vectors that distinguish parts or all of an open reading frame (ORF) and its uses in vaccine development and antibody production.

2. Description of Related Art

Progress in functional genomics is currently hampered on a practical level by the extremely large number of clones that must be incorporated into a genomic library to ensure that each protein-coding segment is present and cloned in its correct frame and orientation for expression. For a simple virus or bacterium, in which most of the genomic DNA encodes proteins, this corresponds minimally to a 6-fold increase in the size of the library to be screened. This problem is exacerbated when screening eukaryotic genomes, since only a small portion of the DNA contains genes. Consequently, many functional screens of eukaryotic genomes are untenable for reasons of magnitude, particularly those requiring animal models for testing.

In contrast to the small, compact genomes of bacteria and viruses, eukaryotic parasites, for example, have large, complex genomes, typically 30 to 80 Mb, with 5 to 20 percent coding material. Furthermore, they have complex life cycles that involve several stages in two or more hosts, and many can undergo antigenic gene switching to evade the host immune system. Consequently, it has been exceedingly difficult to identify protective antigens, and there are no effective vaccines against most eukaryotic parasites to date. Given that these organisms are responsible for a large number of serious diseases in humans as well as in agriculturally important animals, there is clearly a need for a technological breakthrough to allow prophylactic vaccination against these parasites. Moreover, there exists a general need for vaccines against other pathogens as well.

Several ORF selection vectors have previously been described that are based on fusing DNA inserts to enzymatic reporter genes (reviewed in Weinstock, 1987). Most of these vectors were designed to select ORF-containing DNA fragments from specific single genes as a means of facilitating antibody production (Ruther et al., 1982; Weinstock et al., 1983). A more recent enzyme-based strategy has been to create an ORF-TRAP selection system based on intein splicing (Daugelat and Jacobs, 1999; U.S. Pat. No. 5,981,182). However, one of the main limitations of ORF screens that are predicated on enzymatic activity is that this functional property is likely to be perturbed by many ORF fusions. As a result of this instability, all of the aforementioned ORF vectors suffer from the same major disadvantage in that they do not tolerate a wide repertoire of protein fusions. Consequently, they are not amenable to functional genomic screening.

These ORF selection vectors can be employed in the development of genetic (DNA) immunization (Tang et al., 1992), which provides an unbiased approach to vaccine discovery. A method called expression library immunization (ELI) involves administering a large number of protein vaccine candidates in the form of an expression construct to an animal and determining whether an immune response is elicited (U.S. Pat. No. 5,703,057). Once again, problems of dealing with large genomes in functional genomic methods continue to exist, and ELI is another example of a method that could take advantage of ORF selection vectors.

Therefore, an improved set of vectors that can be used to select ORFs is desirable. The present invention addresses this need by providing ORF selection vectors that can be used in the field of functional genomics, for example, to create vaccines against a wide variety of pathogenic and infectious agents. The invention also provides methods of producing and using such ORF selection vectors.

### SUMMARY OF THE INVENTION

This invention takes advantage of the inventors' success in streamlining functional genomic screens. An efficient screen has been devised for selecting functional open reading frames from complex genomes that contain large amounts of noncoding DNA. To this end, the inventors have designed open reading frame (ORF) selection vectors, such as pORF-GFP, which allows expression of a green fluorescent protein (GFP) reporter gene only when it contains an ORF. In practice, this reduces the number of candidate ORF clones by approximately 95%. Therefore, the present invention comprises compositions and methods involving an ORF selection vector.

Some embodiments of the present invention concern an ORF selection vector that comprises a promoter that is operably linked to a start codon and reporter gene that is positioned downstream from both the promoter and the start codon. In preferred embodiments of the present invention, the reporter gene is out of frame with respect to its normal coding sequence. Consequently, the reporter gene is not expressed unless a nucleic acid sequence is inserted upstream of it, and the inserted sequence is of the proper length (3n+1) and allows the reporter gene to be expressed-that is, there are no stop codons in the segment, or if there is a stop codon, there is a start codon downstream of the stop codon.

In some aspects, the ORF selection vector may be inserted with a nucleic acid sequence between the vector's start codon and the reporter gene. The insertion may position the reporter gene so that it is now in frame and can be properly expressed. In other aspects of the claimed invention, the inserted nucleic acid sequence is genomic DNA. It is contemplated that genomic DNA can be from a eukaryote or a prokaryote. Genomic DNA may also be obtained from a pathogen or a parasite. If genomic DNA is retrieved from a parasite, examples of such parasites include Plasmodium falciparum, Neospora caninum, and Trypanosoma cruzi, though genomic DNA from other parasites is considered within the scope of the invention. It is also contemplated that the genome may be derived from various cells, such as cancer cells or a cells at a particular developmental stage, or otherwise distinguishable.

In some aspects of the invention, the reporter gene lacks a start codon. In other aspects, the reporter gene encodes a gene product that is nonenzymatic, such as a GFP. While in other aspects, the reporter gene is a death gene. The death gene may encode an enzyme, a DNA replication inhibitor, a membrane disrupter, or any other polypeptide that is toxic to a host cell, even if its mechanism of action is unknown. It is contemplated that the origin of such genes may be eukaryotic or prokaryotic, though bacterial death genes are preferred in some aspects of the invention. Such enzymes include barnase, colicin, and SacB. Such DNA replication inhibitors include CcdB, Kid, and GATA. Such membrane disruptors include Hok, holins, or granulysin. Another death-gene encoded gene product is Doc.

As previously mentioned, a nucleic acid sequence may be inserted in the ORF selection vectors of the present invention between a stop codon and a reporter gene. In some aspects of the invention, the inserted nucleic acid sequence is part or all of at least one ORF. It is contemplated that the vector may contain a multiple insert, or it may contain several ORFs, with at least one start codon (also called initiation site or codon) further downstream than a stop codon.

In some embodiments the composition of the present invention have at least one promoter. The promoter may be a eukaryotic or prokaryotic promoter. An example of a prokaryotic promoter that is used in the invention is the T7 promoter, which is well known to those of skill in the art. In still further embodiments, there is at least one restriction endonuclease site between the start codon and the reporter gene. Also, there may be restriction endonuclease sites throughout the vector. The vector may also contain an origin of replication that is derived from either a prokaryotic or eukaryotic organism.

Compositions of the invention also include an ORF selection vector that contains a selectable marker. The marker may be either prokaryotic or eukaryotic in origin. In preferred embodiments, the marker is in frame and expressed to confer antibiotic resistance on a host cell.

Other embodiments of the claimed invention include methods involving the ORF selection vectors. It is contemplated that all of the embodiments relevant to the ORF selection vectors may be employed in the context of all the methods and kits of the present invention.

Methods of producing an ORF selection vector are included and they comprise (a) contacting genomic DNA with at least one restriction endonuclease; (b) obtaining an ORF selection vector according to any of the embodiments or combination of embodiments described above; (c) contacting the ORF selection vector with at least one restriction endonuclease; and, (d) ligating a genomic restriction endonuclease DNA fragment generated from step (a) with the linearized ORF selection vector. It is contemplated that contacting DNA with a restriction endonuclease is under conditions to effect specific digestion of the DNA depending on the particular endonuclease employed.

Methods of producing an ORF selection vector may also include the step of transfecting a host cell with at least one ORF selection vector that contains at least a part of the genomic DNA. The host cell may be eukaryotic or prokaryotic. In some aspects of the invention, the host cell is a bacterial host cell.

In further aspects of the present invention, the ligated ORF selection vector is capable of expressing at least one, if not two reporter genes that it contains. Particularly, it is contemplated that the vector can express a reporter gene that was not previously capable of being expressed by the parent vector (vector from step (b) that does not have inserted genomic DNA).

The genomic restriction endonuclease DNA fragment may comprise a portion of at least one ORF. Multiple fragments are also contemplated to be ligated into the ORF selection vector. Once again, the embodiments described for the vector compositions may be employed with the methods of the claimed invention.

In other aspects of the claimed methods, the restriction endonuclease contacted with the genomic DNA creates a site compatible with the site created by the restriction endonuclease contacted with the ORF selection vector. It is also contemplated that the expression vector is contacted with a phosphatase after it is contacted with a restriction endonuclease.

The invention also covers methods of identifying at least a portion of an ORF comprising (a) contacting genomic DNA with at least one restriction endonuclease; (b) obtaining an ORF selection vector described above; (c) contacting the ORF selection vector with at least one restriction endonuclease; (d) ligating a genomic restriction endonuclease DNA fragment generated from step (a) with the linearized ORF selection vector; (e) transfecting a host cell with the ligated selection vector; (f) determining whether the reporter gene is expressed. The permutations of the compositions and methods described above can be practiced with these methods of identifying ORFs as well.

Similarly, these various embodiments can also be practiced with the methods of the present invention related to inducing an immune response in an animal. In some embodiments, this comprises: (a) obtaining an ORF selection vector; (b) identifying an ORF by determining whether the reporter gene is expressed; (c) if the reporter gene is expressed, subcloning the ORF into an expression construct lacking the reporter gene; and (d) introducing the expression construct into an the animal in a manner effective to induce an immune response against one or more antigens that may be encoded by the construct.

In some embodiments, the promoter contained with the ORF selection vector is a eukaryotic promoter that is from the same species as the animal. That is a mouse promoter may be used when the ORF selection vector is administered to a mouse, for example.

The methods may also further include testing the animal for an immune response. A wide variety of assays are available including the animal challenge model. This test can involve challenging the animal with an expression product of the ORF.

In further embodiments, another step of the method includes obtaining antibodies generated in response to one or more antigens encoded by the introduced second construct.

Other methods of the invention including preparing an antigen including the following steps: (a) obtaining an ORF selection vector; (b) identifying an ORF by determining whether the reporter gene is expressed; (c) if the reporter gene is expressed, subcloning the ORF into an expression construct lacking the reporter gene; (d) administering to an animal a pharmaceutical composition comprising one or more expression constructs; and (e) identifying the antigen or antigens so expressed.

Moreover, the invention comprises kits involving or related to the compositions and methods described above. Included are kits for identifying an antigen that include (a) an ORF selection vector. It further embodiments, the kit also includes an expression construct lacking the reporter gene.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: pORF-GFP plasmid map. In addition to a GFP reporter gene, the ORF selection vector contains: 1) an ATG start codon positioned out of frame with respect to the GFP gene, 2) an IPTG inducible T7 promoter to drive bacterial expression, 3) and a BamHI cloning site located between the initiating ATG and the start of the GFP gene which may be used to insert Sau3A-digested pathogen DNA.

FIG. 2: pORF-GFP Transcription/translation regulatory region. Transcription of cloned DNA is under the control of a strong T7 promoter. Translation initiates from an ATG codon that is located immediately upstream of a unique BamHI cloning site. The initiating ATG is out of frame with respect to the ATG of the downstream GFP reporter gene.

FIG. 3: pORF-PBA-GFP transcription/translation regulatory region. Transcription of cloned DNA is under the control of a strong T7 promoter. Translation initiates from an ATG codon that is located immediately upstream of a unique BamHI cloning site. The BamHI cloning site is spanned by restriction sites for PacI and AscI. The natural ATG of GFP has been substituted with a GCG codon for alanine.

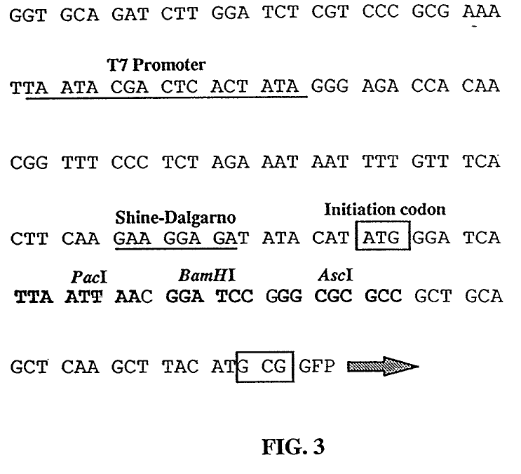

FIG. 4: pORF-PBA-GFP transcription/translation regulatory region. Transcription of cloned DNA is under the control of a strong T7 promoter. Translation initiates from an ATG codon that is located immediately upstream of a unique NarI cloning site. The NarI cloning site is spanned by restriction sites for PacI and AscI. The natural ATG of GFP has been substituted with a GCG codon for alanine.

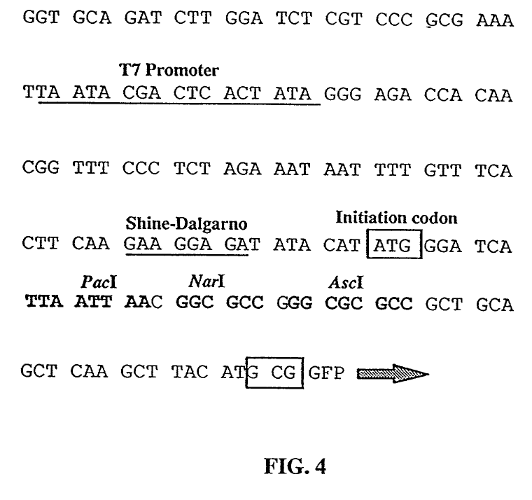

FIG. 5: GORF and STORF distribution of P. faliparum (chrom. 1II and III). The frequency of GORFs (gene ORFs) show the number of DNA fragments of a particular length that correspond to protein-coding DNA. The frequency of STORFs shows the number of fragments that fall between two stop codons and that do not encode proteins.

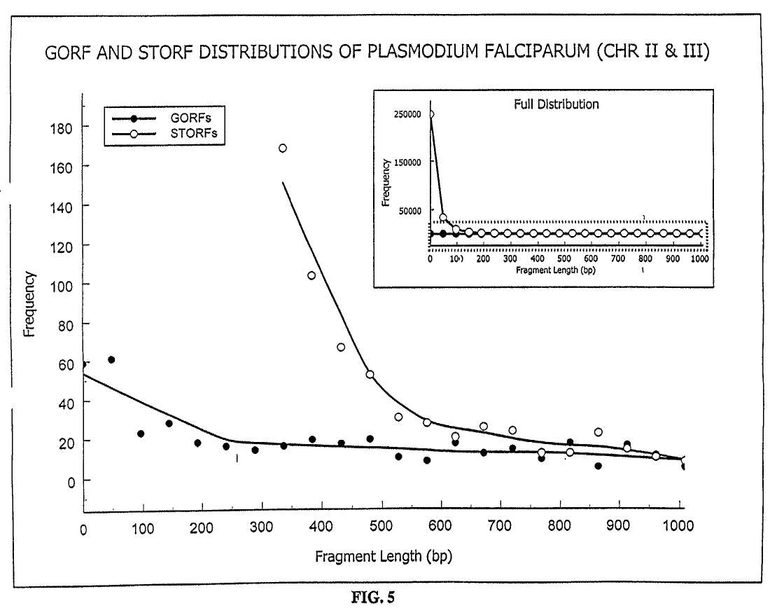

FIG. 6: pORF-FINDER1. Modified pORF-GFP plasmid in which the first ATG of GFP is removed to reduce the incidence of false positives. To increase the stability of fusion proteins, an alanine rich region is included immediately upstream of GFP. To allow the direct excision of inserts, PacI and AscI sites flank the BamHI site.

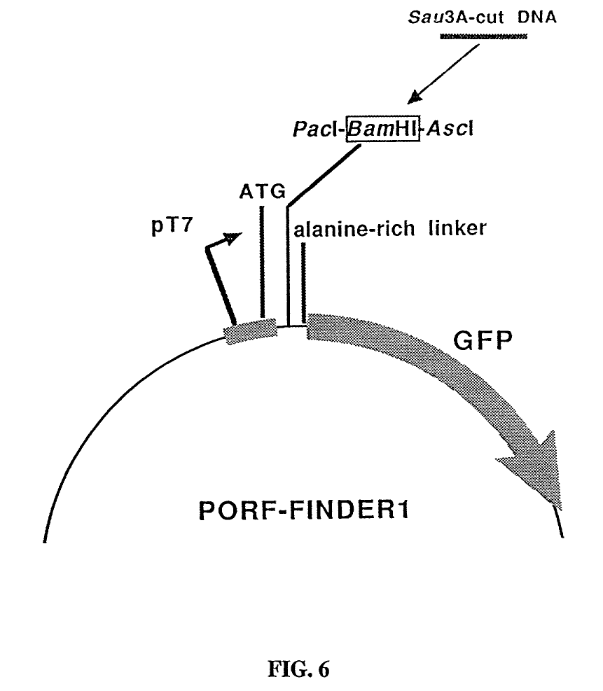

FIG. 7: pORF-FINDER2. Vector pORF-FINDER2 is identical to pORFFINDER1 (FIG. 6) except that a NarI site replaces the BamHI site. The NarI site is compatible with DNA that has been digested with TaqI, MaeI, MspI, AciI, and HinP1I.

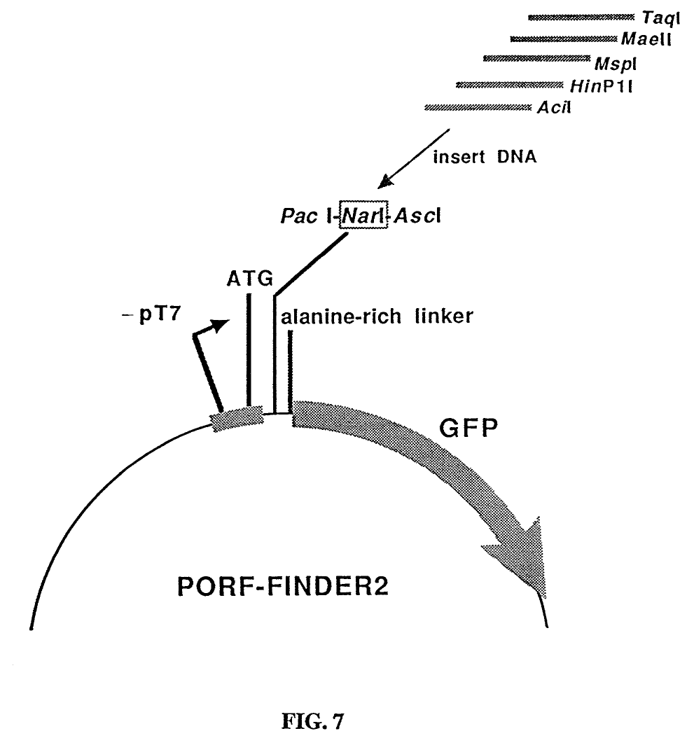

FIG. 8: Use of ORF selection to select plasmids for use in ELI of Neospora caninum genomic DNA. Using the optimized pORF-FINDER vectors and predicted insert size range, three separate libraries were prepared with Sau3A-, MaeII- or TaqI- partially digested DNA from the parasite N. caninum. A total of 42,000 ORF-ontaining clones (approximately one genome equivalent) were isolated for ELI testing. The entire ORF screening procedure is represented.

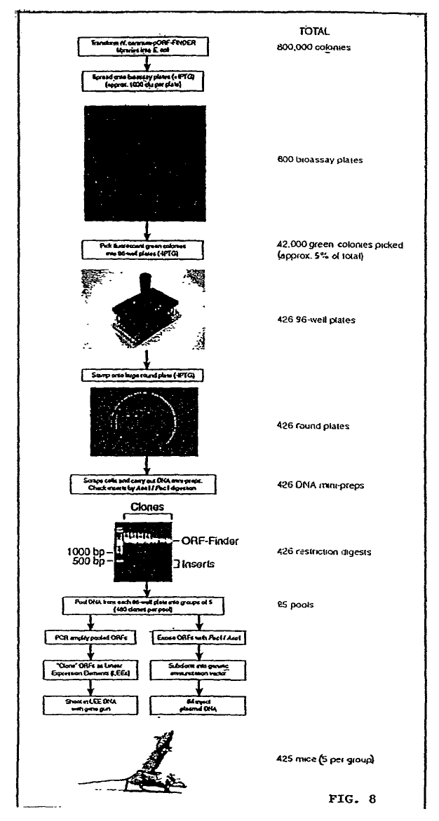

### DESCRIPTION OF ILLUSTRATIVE EMBODIMENTS

As previously discussed, ORF selection vectors have proven less than optimal thus far. The inventors have two strategies to address this problem: 1) a differential selection ORF vector that utilizes a nonenzymatic reporter gene, the green fluorescent protein (GFP), and 2) a positive selection ORF vector that utilizes a death gene to eliminate non-ORF fusions. To test the first strategy, the inventors constructed an ORF selection vector that contains the GFP reporter gene (pORF-GFP). GFP was chosen for our ORF selection system because it is an unusually stable protein which is very tolerant of fusions (Prasher, 1995; Cubitt et al., 1995; Tsien, 1998). To increase the stability and detection of ORF-GFP fusion proteins, the inventors used a version of GFP that has undergone directed evolution to enhance these properties (Crameri et al., 1996). To determine the efficacy of this differential selection system, the inventors used pORF-GFP to construct libraries of total genomic DNA from a eukaryote (Saccharomyces cerevisiae). The inventors observed that approximately 5% of the colonies were fluorescent, as predicted, and most of the inserts were indeed ORFs. Given that this primary genomic screen is carried out in bacteria, the outcome is a relatively rapid and inexpensive en masse ORF selection for any eukaryotic genome. More importantly, it significantly reduces the size of any downstream functional screens, which are typically labor intensive and costly. As an extension of this work, the inventors have carried out screens of genomic DNA from two eukaryotic parasites (Neospora caninum and Trypanosoma cruzi) and have shown that pORF-GFP does indeed allow ORF selection from these complex genomes. These experiments demonstrate the feasibility of the pORF-GFP selection system. It is contemplated that the vector compositions of the present invention can be employed in a variety of methods, including genetic immunization protocols such as expression library immunization (ELI) in the development of vaccines against potentially any agent that contains genomic sequences.

A. Nucleic Acids

Compositions of the present invention include expression constructs and ORF selection vectors that are encoded by a nucleic acid molecule. An “expression construct” refers to a vector that is capable of expressing part or all of at least one open reading frame (ORF). An “ORF selection vector” refers to a particular type of expression construct that is capable of allowing for the identification of part or all of at least one ORF. In some embodiments of the present invention, an ORF selection vector contains a reporter gene that is expressed only in the presence of at least a part of all or one ORF inserted upstream of the gene.

Genes are sequences of DNA in an organism's genome encoding information that is converted into various products making up a whole cell. They are expressed by the process of transcription, which involves copying the sequence of DNA into RNA. Most genes encode information to make proteins, but some encode RNAs involved in other processes. If a gene encodes a protein, its transcription product is called “messenger” RNA (mRNA). After transcription in the nucleus (where DNA is located), the mRNA must be transported into the cytoplasm for the process of translation, which converts the code of the mRNA into a sequence of amino acids to form protein.

In certain aspect, the present invention concerns the isolation of nucleic acid from a cell. When nucleic acid is isolated from a cell, it is specifically contemplated that the nucleic acid isolated will be genomic DNA. For the purpose of the instant invention, genomic DNA is considered to be DNA derived from the chromosome or chromosomes of the host cell. As used herein “isolated nucleic acid” refers to a nucleic acid that has been isolated free of, or is otherwise free of, bulk of cellular components and macromolecules such as lipids, proteins, small biological molecules, and the like. As different species may have a RNA or a DNA containing genome, the term “isolated nucleic acid” encompasses both the terms “isolated DNA” and “isolated RNA.” Thus, the isolated nucleic acid may comprise a RNA or DNA molecule isolated from, or otherwise free of, the bulk of total RNA, DNA or other nucleic acids of a particular species. As used herein, an isolated nucleic acid isolated from a particular species is referred to as a “species-specific nucleic acid.” When designating a nucleic acid isolated from a particular species, such as human, such a type of nucleic acid may be identified by the name of the species. For example, a nucleic acid isolated from one or more humans would be an “isolated human nucleic acid.”

Of course, more than one copy of an isolated nucleic acid may be isolated from biological material, or produced in vitro, using standard techniques that are known to those of skill in the art. In particular embodiments, the isolated nucleic acid is assayed for its ability to express a protein, polypeptide or peptide.

In certain embodiments, a “gene” refers to a nucleic acid that is transcribed. In some cases, a gene may be transcribed and then translated to produce a “gene product.” As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. As used herein, the term open reading frame refers to a length of DNA or RNA sequence capable of being translated into a peptide normally located between a start or initiation signal and a termination signal. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the open reading frame of interest, forms the significant part of the coding region of the isolated nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

In certain embodiments, the open reading frame is a nucleic acid segment. As used herein, the term “nucleic acid segment,” are smaller fragments of a nucleic acid, such as for non-limiting example, those that encode only part of the gene and/or gene peptide or polypeptide sequence. Thus, a “nucleic acid segment” may comprise any part of the open reading frame of the gene sequence(s) from about 19 nucleotides to the full length of the peptide or polypeptide encoding region.

As used herein in particular embodiments of the invention, a nucleic acid segment or DNA fragment will be understood to include a contiguous nucleic acid sequence of about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 390, about 400, about 450, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3300, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, or about 5600 nucleotides or so.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on. In certain embodiments, the nucleic acid segment may be a probe or primer.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

B. Detection of Nucleic Acids

1. Oligonucleotide Probes and Primers

As compositions comprising nucleic acid sequences and methods of effecting protein expression are included in the present invention, it is contemplated that nucleic acid-based assays, uses, and detection methods are useful in the context of the invention.

Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of annealing to the nucleic acid segment being described under relatively stringent conditions such as those described herein.

Primers should be of sufficient length to provide specific annealing to a RNA or DNA tissue sample. The use of a primer of between about 10-14, 15-20, 21-30 or 31-40 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained.

Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 300, 500, 600, 700, 800, and longer are contemplated as well. Accordingly, nucleotide sequences may be selected for their ability to selectively form duplex molecules with complementary stretches of genes or RNAs or to provide primers for amplification of DNA or RNA from cells, cell lysates and tissues. The method of using probes and primers of the present invention is in the selective amplification and detection of genes, changes in gene expression, gene polymorphisms, single nucleotide polymorphisms, changes in mRNA expression wherein one could be detecting virtually any gene or genes of interest from any species. The target polynucleotide will be RNA molecules, mRNA, cDNA, DNA or amplified DNA. By varying the stringency of annealing, and the region of the primer, different degrees of homology may be discovered.

The particular amplification primers of the present invention will be specific oligonucleotides which encode particular features including the recognition site for frequently cutting restriction enzymes, primer sequences, and degenerate sequences of 3, 4, 5, 6, 7, 8 or more consecutive bases to ensure amplification of all target genes. Generally, the present invention may involve the use of a variety of other PCR™ primers which hybridize to a variety of other target sequences.

Amplification primers may be chemically synthesized by methods well known within the art (Agrawal, 1993). Chemical synthesis methods allow for the placement of detectable labels such as fluorescent labels, radioactive labels etc. to be placed virtually anywhere within the polynucleic acid sequence. Solid phase method of synthesis also may be used.

The amplification primers may be attached to a solid-phase, for example, a latex bead; or the surface of a chip. Thus, the amplification carried out using these primers will be on a solid support/surface.

Furthermore, some primers of the present invention will have a recognition moiety attached. A wide variety of appropriate recognition means are known in the art, including fluorescent labels, radioactive labels, mass labels, affinity labels, chromophores, dyes, electroluminescence, chemiluminescence, enzymatic tags, or other ligands, such as avidin/biotin, or antibodies, which are capable of being detected and are described below.

2. Amplification

a. PCR™

In some embodiments, poly-A mRNA is isolated and reverse transcribed (referred to as RT) to obtain cDNA which is then used as a template for polymerase chain reaction (referred to as PCR™) based amplification. In other embodiments, cDNA may be obtained and used as a template for the PCR™ reaction. In PCR™, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred.

The primers are used in any one of a number of template dependent processes to amplify the target-gene sequences present in a given template sample. One of the best known amplification methods is PCR™ which is described in detail in U.S. Pat. Nos. 4,683,195, 4,683,202 and 4,800,159, each incorporated herein by reference.

In PCR™, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates are added to a reaction mixture along with a DNA polymerase, e.g., Taq polymerase, that facilitates template-dependent nucleic acid synthesis.

If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles”, are conducted until a sufficient amount of amplification product is produced.

Next, the amplification product is detected. In certain applications, the detection may be performed by visual means. Alternatively, the detection may involve indirect identification of the product via fluorescent labels, chemiluminescence, radioactive scintigraphy of incorporated radiolabel or incorporation of labeled nucleotides, mass labels or even via a system using electrical or thermal impulse signals (Affymax technology).

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641, filed Dec. 21, 1990.

b. LCR

Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

C. Qbeta Replicase

Qbeta Replicase, described in PCT Patent Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which can then be detected.

d. Isothermal Amplification

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. 1992, incorporated herein by reference.

e. Strand Displacement Amplification

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

f. Cyclic Probe Reaction

Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

g. Transcription-Based Amplification

Other nucleic acid amplification procedures include transcription-based amplification systems (TAS), including nucleic acid sequence based amplification (NASBA) and 3SR, Kwoh et al., 1989; PCT Patent Application WO 88/10315 et al., 1989, each incorporated herein by reference).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

h. Other Amplification Methods

Other amplification methods, as described in British Patent Application No. GB 2,202,328, and in PCT Patent Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Davey et al., European Patent Application No. 329,822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention, Wu et al., 1989, incorporated herein by reference).

2. Restriction Enzymes

Restriction-enzymes recognize specific short DNA sequences four to eight nucleotides long (see Table 1), and cleave the DNA at a site within this sequence. In the context of the present invention, restriction enzymes are used to cleave DNA molecules at sites corresponding to various restriction-enzyme recognition sites. The list below provides an example of specific restriction enzymes that may be used in the invention.

TABLE 1
RESTRICTION ENZYMES

| Enzyme | Recognition |
|---|---|
| Name | Sequence |
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| BciV I | GTATCC |
| Bcl I | TGATCA |
| Bfa I | CTAG |
| Bgl I | GCCNNNNNGGC |
| Bgl II | AGATCT |
| Blp I | GCTNAGC |
| Bmr I | ACTGGG |
| Bpm I | CTGGAG |
| BsaA I | YACGTR |
| BsaB I | GATNNNNATC |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| BsmA I | GTCTC |
| BsmB I | CGTCTC |
| BsmF I | GGGAC |
| Bsm I | GAATGC |
| BsoB I | CYCGRG |
| Bsp1286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAP I | GCANNNNNTGC |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dde I | CTNAG |
| Dpn I | GATC |
| Dpn II | GATC |
| Dra I | TTTAAA |
| Dra III | CACNNNGTG |
| Drd I | GACNNNNNNGTC |
| Eae I | YGGCCR |
| Eag I | CGGCCG |
| Ear I | CTCTTC |
| Eci I | GGCGGA |
| EcoN I | CCTNNNNNAGG |
| EcoO109 I | RGGNCCY |
| EcoR I | GAATTC |
| EcoR V | GATATC |
| Fau I | CCCGCNNNN |
| Fnu4H I | GCNGC |
| Fok I | GGATG |
| Fse I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind Ill | AAGCTT |
| Hinf I | GANTC |
| HinP1 I | GCGC |
| Hpa I | GTTAAC |
| Hpa II | CCGG |
| Hph I | GGTGA |
| Kas I | GGCGCC |
| Kpn I | GGTACC |
| MaeII | ACGT |
| Mbo I | GATC |
| Mbo II | GAAGA |
| Mfe I | CAATTG |
| Mlu I | ACGCGT |
| Mly I | GAGTCNNNNN |
| Mnl I | CCTC |
| Msc I | TGGCCA |
| Mse I | TTAA |
| Msl I | CAYNNNNRTG |
| MspA1 I | CMGCKG |
| Msp I | CCGG |
| Mwo I | GCNNNNNNNGC |
| Nae I | GCCGGC |
| Nar I | GGCGCC |
| Nci I | CCSGG |
| Nco I | CCATGG |
| Nde I | CATATG |
| NgoMI V | GCCGGC |
| Nhe I | GCTAGC |
| Nla III | CATG |
| Nla IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| PleI | GAGTC |
| Pme I | GTTTAAAC |
| Pml I | CACGTG |
| PpuM I | RGGWCCY |
| PshA I | GACMNNNGTC |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| ScrF I | CCNGG |
| SexA I | ACCWGGT |
| SfaN I | GCATC |
| Sfc I | CTRYAG |
| Sfi I | GGCCNNNNNGGCC |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Taq I | TCGA |
| Tfi I | GAWTC |
| Tli I | CTCGAG |
| Tse I | GCWGC |
| Tsp45 I | GTSAC |
| Tsp509 I | AATT |
| TspR I | CAGTG |
| Tth111 I | GACNNNGTC |
| Xba I | TCTAGA |
| Xcm I | CCANNNNNNNNNTGG |
| Xho I | CTCGAG |
| Xma I | CCCGGG |
| Xmn I | GAANNNNTTC |

4. Other Enzymes

Other enzymes that may be used in conjunction with the invention include nucleic acid modifying enzymes listed in the following tables.

TABLE 2
POLYMERASES AND REVERSE TRANSCRIPTASES

Thermostable DNA Polymerases:
OmniBase ™ Sequencing Enzyme
Pfu DNA Polymerase
Taq DNA Polymerase
Taq DNA Polymerase, Sequencing Grade
TaqBead ™ Hot Start Polymerase
AmpliTaq Gold
Tfl DNA Polymerase
Tli DNA Polymerase
Tth DNA Polymerase
DNA Polymerases:
DNA Polymerase I, Klenow Fragment, Exonuclease Minus
DNA Polymerase I
DNA Polymerase I Large (Klenow) Fragment
Terminal Deoxynucleotidyl Transferase
T4 DNA Polymerase
Reverse Transcriptases:
AMV Reverse Transcriptase
M-MLV Reverse Transcriptase

TABLE 3
DNA/RNA MODIFYING ENZYMES

Ligases:
T4 DNA Ligase
Alkaline Phosphatases
Calf Intestinal Alkaline Phosphatase (CIP)

5. Labels

Recognition moieties incorporated into primers, incorporated into the amplified product during amplification, or attached to probes are useful in identification of the amplified molecules. A number of different labels may be used for the purpose such as fluorophores, chromophores, radio-isotopes, enzymatic tags, antibodies, chemiluminescence, electroluminescence, affinity labels, etc. One of skill in the art will recognize that these and other fluorophores not mentioned herein can also be used with success in this invention.

Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

Examples of enzyme tag include enzymes such as such as urease, alkaline phosphatase or peroxidase to mention a few and colorimetric indicator substrates can be employed to provide a detection means visible to the human eye or spectrophotometrically, to identify specific hybridization with complementary nucleic acid-containing samples. All these examples are generally known in the art and the skilled artisan will recognize that the invention is not limited to the examples described above.

The following fluorophores are specifically contemplated to be useful in practicing the present invention. Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

C. Nucleic Acid-Based Expression Systems

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

2. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that downstream sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant and/or heterologous promoter, which refers to a promoter that is not normally associated with a nucleic acid sequence in its natural environment. A recombinant and/or heterologous enhancer refers also to an enhancer not normally associated with a nucleic acid sequence in its natural environment. Such promoters or enhancers may include promoters or enhancers of other genes, and/or promoters or enhancers isolated from any other prokaryotic, viral, and/or eukaryotic cell, and/or promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and/or cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Tables 3 lists several elements/promoters that may be employed, in the context of the present invention, to regulate the expression of a gene. This list is not intended to be exhaustive of all the possible elements involved in the promotion of expression but, merely, to be exemplary thereof. Table 4 provides examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al, 1986; Fujita et al., 1987; Goodborn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Kiamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al, 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Chol et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | poly(rl)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

3. Initiation Signals and Internal Ribosome Binding Sites

A specific initiation signal also will be required for efficient translation of coding sequences. These signals include the ATG initiation codon and/or adjacent sequences. Exogenous translational control signals, including the ATG initiation codon, may need to be provided. One of ordinary skill in the art would readily be capable of determining this and/or providing the necessary signals. It is well known that the initiation codon must be “in-frame” with the reading frame of the desired coding sequence to ensure translation of the entire insert. The exogenous translational control signals and/or initiation codons can be either natural and/or synthetic. It is contemplated that start codons for the purpose of the instant invention may be located downstream from a stop codon and still function for the purpose contemplated by the inventors of initiating translation.

The efficiency of expression may be enhanced by the inclusion of appropriate transcription enhancer elements. The region upstream of the initiation site may also be engineered to include a Shine Dalgamo sequence, CAAT box, TATA box or other upstream transcription or translation enhancement element or ribosomal binding site commonly known to those of ordinary skill.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. No. 5,925,565 and 5,935,819, herein incorporated by reference).

4. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

5. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

6. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

7. Reporters

The present invention includes expression constructs and methods of employing expression constructs. In some aspects, the present invention concerns an ORF selection vector. An ORF selection vector of the present invention may include a reporter gene that allows the presence of an ORF to be detected and/or identifies whether the expression construct is present in a cell.

Accordingly, in one embodiment, an ORF selection vector includes a reporter gene that is cloned downstream from an insertion site where genomic DNA is inserted. In some cases, the reporter gene lacks its own start site and is out of frame and consequently, it can be expressed only when the inserted DNA contains an open reading frame and is a length that places the reporter gene in frame (length=3n+1).

In other embodiments of the invention, an expression construct or ORF selection vector contains a reporter gene that identifies which cells contain the vector and/or express the reporter gene that was initially out of frame. Thus expression constructs of the present invention may be identified in vitro or in vivo by including a reporter gene in the expression vector.

When expressed, such reporter genes confer an identifiable change to the cell permitting identification of cells containing an expression vector that permitted the reporter gene to be expressed. Gene products of a reporter gene would include selectable markers, nonselectable markers, and screenable markers. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Selectable markers may be either enzymatic or non-enzymatic. For the purpose of the instant invention, a non-enzymatic marker would confer a property upon the cell that does not result from the catalysis of a reaction by the expressed selectable marker. An example of a nonenzymatic marker is GFP. An example of an enzymatic marker is luciferase. A list of reporters that may be employed is included in Table 5.

TABLE 5
Reporter Genes

Ampicillin resistance
Tetracycline resistance
Kanamycin resistance
Streptomycin resistance
Zeocin resistance
β-gal
GFP
Luciferase

Usually the inclusion of a drug selection marker aids in the cloning and identification of transformants, for example, genes that confer resistance to neomycin, puromycin, hygromycin, DHFR, GPT, zeocin and histidinol are useful selectable markers. As used herein, a “nonselectable gene” or “nonselectable marker” refers to a nucleic acid sequence that encodes a gene product that does not allow selection, which refers to the use of conditions that allow for the discrimination of cells displaying a required phenotype, for example, resistance to survive in a particular media.

In addition to markers conferring a phenotype that allows for the discrimination of transformants based on the implementation of conditions, other types of markers—screenable markers such as GFP, whose basis is colorimetric analysis—are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. Further examples of selectable and screenable markers are well known to one of skill in the art.

While some embodiments of the present invention use nonselectable and/or non-enzymatic reporter genes such as GFP, in other instances of the claimed invention ORF selection vectors include a reporter gene that is a death (toxin) gene. A death gene encodes a protein that is toxic to its host cell. A large number of “death genes” have been found that kill the bacterial host cell upon expression (reviewed in Bugge and Gerdes, 1995; Santos-Sierra et al., 1997; Gotfredsen and Gerdes, 1998). The bacterial protein degradation signal is an 11 amino acid sequence that signals to the cell to rapidly degrade the expressed protein (Gottesman, 1999).

For example, a bacterial death gene encodes a polypeptide that is toxic to a bacterial cell unless a degradation signal is also expressed in that cell. In this case, the death gene is not strictly a selectable marker because no selective conditions are employed to distinguish cells. Instead, in some embodiments of the invention, the degradation signal is located on the same vector as the death gene. In one aspect, the degradation signal is out of frame and placed downstream of the death gene, with at least restriction endonuclease site between them. The degradation signal can be expressed only if an ORF of the proper length is inserted in front of it.

Examples of gene products encoded by death genes include, but are not limited to, the following classes of proteins: enzymes, DNA replication inhibitors, and membrane disruptors. A death gene can encode an enzyme such as: barnase, which is an RNase (Yazynin et al., 1999 and references therein); colicin, which is an E3 RNase that cuts the 16srRNA (Diaz et al., 1994 and references therein); and SacB, which is a levan sucrase (Pelicic et al., 1996; Recorbet et al., 1999 and references therein both). DNA replication inhibitors encoded by death genes include: CcdB, which poisons DNA gyrase (Jensen et al., 1995 and references therein); Kid, which inhibits initiation of DNA replication (Ruiz Echevarria et al., 1995 and references therein); and GATA, which inhibits initiation of DNA replication (Trudel et al., 1996 and references therein). Gene products of death genes that disrupt the membrane include: Hok, which interferes with cell membranes (Gultyaev et al., 1997 and references therein); holins, which creates pores in the inner cell membrane of a bacterium (Young, 1992 and references therein); and granulysin, which creates pores in bacterial membranes (Stenger et al., 1998 and references therein). Other nucleic acid-encoded agents that are toxic to a cell are also contemplated in the context of the present invention, such as Doc, whose mechanism is unknown (Lehnherr et al., 1995 and references therein).

D. DNA Delivery Using a Viral Vector

In some embodiments the compositions of the present invention are introduced into a cell to practice methods of the invention. Numerous methods exist for introducing exogenous DNA into a cell, some of which are described below. One of ordinary skill in the art is familiar with such techniques and the dosages and route of administration necessary to achieve the delivery of nucleic acids molecules.

The ability of certain viruses to infect cells or enter cells via receptor-mediated endocytosis and to integrate into host cell genome and express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

1. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). The typical vector according to the present invention is replication defective and will not have an adenovirus E1 region.

Adenovirus is particularly suitable for use as a gene transfer vector because of its mid-sized genome, ease of manipulation, high titer, wide target-cell range and high infectivity. In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication-deficient, depend on a unique helper cell line, designated 293, which was transformed from human embryonic kidney cells by Ad5 DNA fragments and constitutively expresses E1 proteins (E1A and E1B; Graham et al., 1977). Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

2. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin, et al., 1984; Laughlin, et al., 1986; Lebkowski, et al., 1988; McLaughlin, et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt, et al., 1994; Lebkowski, et al., 1988; Samulski, et al., 1989; Yoder, et al., 1994; Zhou, et al., 1994; Hermonat and Muzyczka, 1984; Tratschin, et al., 1985; McLaughlin, et al., 1988) and genes involved in human diseases (Flotte, et al., 1992; Luo, et al., 1994; Ohi, et al., 1990; Walsh, et al., 1994; Wei, et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski, et al., 1989; McLaughlin, et al., 1988; Kotin, et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

4. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

5. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

6. Other Methods of DNA Delivery

In various embodiments of the invention, DNA is delivered to an animal as an expression construct. In order to effect expression of a gene construct, the expression construct must be delivered into a cell. As described herein, a mechanism for DNA delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane. Some of these techniques may be successfully adapted for in vivo or ex vivo use, as discussed below.

a. Liposome-Mediated Transfection

In a farther embodiment of the invention, the expression construct may be entrapped in a liposome. Liposomes are vesicular structures characterized by a phospholipid bilayer membrane and an inner aqueous medium and are discussed in section 4.5.2. Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

b. Electroporation

In certain embodiments of the present invention, the expression construct is introduced into the cell via electroporation. Electroporation involves the exposure of a suspension of cells and DNA to a high-voltage electric discharge. Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

C. Calcium Phosphate Precipitation or DEAE-Dextran Treatment

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

e. Direct Microinjection or Sonication Loading

Further embodiments of the present invention include the introduction of the expression construct by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

f. Adenoviral-Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

g. Receptor Mediated Transfection

Still further expression constructs that may be employed to deliver the tissue-specific promoter and transforming construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993; incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene-targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

E. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and or/expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector and/or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials. (www.atcc.org) An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurrat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and/or their cognate polypeptides, proteins, or peptides.

F. Separation and Quantitation Methods

As compositions and methods of the present invention involve cloning and subcloning nucleic acid fragments, it may be desirable to separate nucleic acid molecules of several different lengths. For example, candidate ORF segments in a particular size range may be inserted into ORF selection vectors.

1. Gel Electrophoresis

In one embodiment, nucleic acid molecules are separated by agarose, agarose-acrylamide or polyacrylamide gel electrophoresis using standard methods (Sambrook et al., 1989).

2. Chromatographic Techniques

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982). In yet another alternative, labeled cDNA products, such as biotin or antigen can be captured with beads bearing avidin or antibody, respectively.

3. Microfluidic Techniques

Microfluidic techniques include separation on a platform such as microcapillaries, designed by ACLARA BioSciences Inc. or the LabChip™ “liquid integrated circuits” made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR™ apparatus for collection and amplification of nucleic acids from a specimen. U.S. Pat. Nos. 5,304,487 and 5,296,375, discuss devices for collection and analysis of cell containing samples and are incorporated herein by reference. U.S. Pat. No. 5,856,174 describes an apparatus which combines the various processing and analytical operations involved in nucleic acid analysis and is incorporated herein by reference.

4. Capillary Electrophoresis

In some embodiments, it may be desirable to provide an additional, or alternative means for analyzing the amplified genes. In these embodiments, micro capillary arrays are contemplated to be used for the analysis.

Microcapillary array electrophoresis generally involves the use of a thin capillary or channel that may or may not be filled with a particular separation medium. Electrophoresis of a sample through the capillary provides a size based separation profile for the sample. The use of microcapillary electrophoresis in size separation of nucleic acids has been reported in, for example, Woolley and Mathies, 1994. Microcapillary array electrophoresis generally provides a rapid method for size-based sequencing, PCR™ product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, for example, Jacobsen et al., 1994; Effenhauser et al., 1994; Harrison et al., 1993; Effenhauser et al., 1993; Manz et al., 1992; and U.S. Pat. No. 5,904,824, here incorporated by reference. Typically, these methods comprise photolithographic etching of micron scale channels on a silica, silicon or other crystalline substrate or chip, and can be readily adapted for use in the present invention. In some embodiments, the capillary arrays may be fabricated from the same polymeric materials described for the fabrication of the body of the device, using the injection molding techniques described herein.

Tsuda et al., 1990, describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

G. Identification Methods

Nucleic acids may be visualised in order to determine concentration or size. One typical visualization method involves staining of a gel with for example, a flourescent dye, such as ethidium bromide or Vistra Green and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly, using a nucleic acid probe. Following separation of nucleic acids, a labeled, nucleic acid probe is brought into contact with the nucleic acid molecule. The probe preferably is conjugated to a chromophore but may be radiolabeled. In another embodiment, the probe is conjugated to a binding partner, such as an antibody or biotin, where the other member of the binding pair carries a detectable moiety. In other embodiments, the probe incorporates a fluorescent dye or label. In yet other embodiments, the probe has a mass label that can be used to detect the molecule amplified. Other embodiments also contemplate the use of Taqman™ and Molecular Beacon™ probes. In still other embodiments, solid-phase capture methods combined with a standard probe may be used as well.

When using capillary electrophoresis, microfluidic electrophoresis, HPLC, or LC separations, either incorporated or intercalated fluorescent dyes are used to label and detect the nucleic acid molecules. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, primers for the PCR™ can be labeled with a fluorophore, a chromophore or a radioisotope, or by associated enzymatic reaction. Enzymatic detection involves binding an enzyme to primer, e.g., via a biotin:avidin interaction, following separation of nucleic acid molecules on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. A number of the above separation platforms can be coupled to achieve separations based on two different properties.

It is also envisioned that nucleic acids may be sequenced for further identification. Sanger dideoxy-termination sequencing is the means commonly employed to determine nucleotide sequence. The Sanger method employs a short oligonucleotide or primer that is annealed to a single-stranded template containing the DNA to be sequenced. The primer provides a 3′ hydroxyl group that allows the polymerization of a chain of DNA when a polymerase enzyme and dNTPs are provided. The Sanger method is an enzymatic reaction that utilizes chain-terminating dideoxynucleotides (ddNTPs). ddNTPs are chain-terminating because they lack a 3′-hydroxyl residue which prevents formation of a phosphodiester bond with a succeeding deoxyribonucleotide (dNTP). A small amount of one ddNTP is included with the four conventional dNTPs in a polymerization reaction. Polymerization or DNA synthesis is catalyzed by a DNA polymerase. There is competition between extension of the chain by incorporation of the conventional dNTPs and termination of the chain by incorporation of a ddNTP.

Although a variety of polymerases may be used, the use of a modified T7 DNA polymerase (Sequenase™) was a significant improvement over the original Sanger method (Sambrook et al., 1988; Hunkapiller, 1991). T7 DNA polymerase does not have any inherent 5′-3′ exonuclease activity and has a reduced selectivity against incorporation of ddNTP. However, the 3′-5′ exonuclease activity leads to degradation of some of the oligonucleotide primers. Sequenase™ is a chemically-modified T7 DNA polymerase that has reduced 3′ to 5′ exonuclease activity (Tabor et al., 1987). Sequenase™ version 2.0 is a genetically engineered form of the T7 polymerase that completely lacks 3′ to 5′ exonuclease activity. Sequenase™ has a very high processivity and high rate of polymerization. It can efficiently incorporate nucleotide analogs such as dITP and 7-deaza-dGTP, which are used to resolve regions of compression in sequencing gels. In regions of DNA containing a high G+C content, Hoogsteen bond formation can occur which leads to compressions in the DNA. These compressions result in aberrant migration patterns of oligonucleotide strands on sequencing gels. Because these base analogs pair weakly with conventional nucleotides, intrastrand secondary structures during electrophoresis are alleviated. In contrast, Klenow does not incorporate these analogs as efficiently.

The use of Taq DNA polymerase and mutants thereof is a more recent addition to the improvements of the Sanger method (U.S. Pat. No. 5,075,216). Taq polymerase is a thermostable enzyme that works efficiently at 70-75° C. The ability to catalyze DNA synthesis at elevated temperature makes Taq polymerase useful for sequencing templates which have extensive secondary structures at 37° C. (the standard temperature used for Klenow and Sequenase™ reactions). Taq polymerase, like Sequenase™, has a high degree of processivity and like Sequenase 2.0, it lacks 3′ to 5′ nuclease activity. The thermal stability of Taq and related enzymes (such as Tth and Thermosequenase™) provides an advantage over T7 polymerase (and all mutants thereof) in that these thermally stable enzymes can be used for cycle sequencing, which amplifies the DNA during the sequencing reaction, thus allowing sequencing to be performed on smaller amounts of DNA. Optimization of the use of Taq in the standard Sanger Method has focused on modifying Taq to eliminate the intrinsic 5′-3′ exonuclease activity and to increase its ability to incorporate ddNTPs to reduce incorrect termination due to secondary structure in the single-stranded template DNA (EP 0 655 506 B1). The introduction of fluorescently-labeled nucleotides has further allowed the introduction of automated sequencing, which further increases processivity.

H. Genomic Immunization

In some embodiments of the present invention, ORF selection vectors are used in conjunction with a genomic immunization protocol called expression library immunization (ELI) technique, which provides a systematic screening of pathogenic genomes for protective epitopes (Tang et al., 1992; Barry et al., 1995). Generally, ELI is a method of generating and identifying effective vaccines as described in U.S. Pat. Nos. 5,989,553 and 5,703,057; Ulmer et al., 1996; Manoutcharian et al., 1998, which are herein specifically incorporated by reference. By reiterative testing of pools of clones in animal infection models, it is possible to isolate single genes that confer protective immunity. Based on this approach, vaccines can be developed from the antigenic determinants that are given to an animal and then evaluated. The composition and methods of the present invention take advantage of the ability to identify ORFS to enrich the pool of potential antigenic determinants that is given to an animal. Consequently, the ORF selection vectors described herein effect a manifold reduction in the number of clones that are administered to an animal and evaluated. This allows ELI to be used with some genomes that were once thought to large to handle as well as to provide a more cost-effective approach to screening.

ELI generally involves introducing into an animal a large number of antigenic determinants encoded by the genome of an organism, such as a pathogen. Typically, the genome of a pathogen is fragmented, ligated into expression vectors, and then an animal is inoculated with the cloned sub-libraries (called “sibs”). A sib refers to a portion of a parental library that may contain members that overlap with other sibs of the same library. As used in the context of the present invention, “sibbing” means partitioning the parental library into sequential subsets. The inoculated animals are then challenged with the pathogen to reveal which animals elicit a protective immune response and consequently which portions of the sib library have a protective effect. Sibbing methods may then be used to identify the individual or combination of plasmids that confer the protection. Based on the results, the identity of the antigenic determinants may be determined, and regardless of this characterization, vaccines based on the vectors may be prepared. Cellular and/or humoral immune responses caused by a particular clone may lead the way to the development of vaccines. For example, monoclonal and polyclonal antibodies against identified immunogens can be produced and administered as vaccines. Furthermore, ELI can be used to generate an antibody response that also has diagnostic and therapeutic uses as well.

The construction of such libraries is well known to those of skill in the art, such as in Maniatis, 1989; Ausubel et al., 1996; Sambrooke, 1989, all of which are herein incorporated by reference. These constructs from the library are then iteratively administered to an animal, which is then monitored for an immune response. The magnitude of this type of experiment, and consequently some of its difficulties, is decreased by the implementation of an ORF selection vector. While cDNA expression libraries can be used with ELI, construction of a cDNA library requires manipulation of RNA, which is more difficult than working with DNA. Also, genomic libraries can be large and increase the amount of screening necessary since many organisms contain genomic DNA that is largely noncoding. The ORF selection vectors of the present invention circumvent such problems.

I. Proteins, Polypeptides, and/or Peptides

In addition to taking advantage of protein expression as an ORF selection parameter, in some embodiments of the present invention, polypeptides, proteins, and peptides expressed by the composition of the instant invention are contemplated to be useful in a variety of ways. For example, determining the immunogenicity of the specific peptide, polypeptide or protein is within the scope of the invention, as is eliciting an immune response, which is a complicated process involving molecules such as peptides, polypeptides, and proteins.

In some aspects, it is contemplated that once a peptide, protein or polypeptide is determined to be immunogenic, it may be expressed and characterized. The present invention thus provides for the production of proteins, polypeptides, and/or peptides. The proteins, peptides or polypeptides may be full length proteins, however, it is generally contemplated that the protein or peptide will be less then full-length proteins, such as individual domains, regions and/or even epitopic peptides. Where less-than-full-length proteins are concerned the preferred moieties will be those containing predicted immunogenic sites and/or those containing the functional domains identified herein.

Encompassed by the invention are proteinaceous segments of relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and/or more preferably, of from about 15 to about 30 amino acids in length and/or also larger polypeptides of from about 51, about 52, about 53, about 54, about 55, about 56, about 57, about 58, about 59, about 60, about 65, about 70, about 75, about 80, about 85, about 90, about 95, about 100, about 110, about 120, about 130, about 140, about 150, about 160, about 170, about 180, about 190, about 200, and/or up to and/or including proteins corresponding to the full-length sequence.

Where the term “substantially purified” is used, this will refer to a composition in which the protein, polypeptide, and/or peptide forms the major component of the composition, such as constituting about 50% of the proteins in the composition and/or more. In preferred embodiments, a substantially purified protein will constitute more than 60%, 70%, 80%, 90%, 95%, 99% and/or even more of the proteins in the composition.

A peptide, polypeptide and/or protein that is “purified to homogeneity,” as applied to the present invention, means that the peptide, polypeptide and/or protein has a level of purity where the peptide, polypeptide and/or protein is substantially free from other proteins and/or biological components. For example, a purified peptide, polypeptide and/or protein will often be sufficiently free of other protein components so that degradative sequencing may be performed successfully.

Various methods for quantifying the degree of purification of proteins, polypeptides, and/or peptides will be known to those of skill in the art in light of the present disclosure. These include, for example, determining the specific protein activity of a fraction, and/or assessing the number of polypeptides within a fraction by gel electrophoresis. Assessing the number of polypeptides within a fraction by SDS/PAGE analysis will often be preferred in the context of the present invention as this is straightforward.

To purify a protein, polypeptide, and/or peptide a natural and/or recombinant composition, proteins, polypeptides, and/or peptides will be subjected to fractionation to remove various contaminants from the composition. In addition to those techniques described in detail herein below, various other techniques suitable for use in protein purification will be well known to those of skill in the art. These include, for example, precipitation with ammonium sulfate, PEG, antibodies and/or the like and/or by heat denaturation, followed by centrifugation; chromatography steps such as ion exchange, gel filtration, reverse phase, hydroxylapatite, lectin affinity and/or other affinity chromatography steps; isoelectric focusing; gel electrophoresis; and/or combinations of such and/or other techniques.

Another example is the purification of the fusion protein using a specific binding partner. Such purification methods are routine in the art. This is exemplified by the generation of glutathione S-transferase fusion proteins, expression in E. coli, and/or isolation to homogeneity using affinity chromatography on glutathione-agarose and/or the generation of a polyhistidine tag on the N- and/or C-terminus of the protein, and/or subsequent purification using Ni-affinity chromatography.

Although preferred for use in certain embodiments, there is no general requirement that protein, polypeptide, and/or peptide always be provided in their most purified state. Indeed, it is contemplated that less substantially purified protein, polypeptide and/or peptide, which are nonetheless enriched relative to the natural state, will have utility in certain embodiments.

Methods exhibiting a lower degree of relative purification may have advantages in total recovery of protein product, and/or in maintaining the activity of an expressed protein. Inactive products also have utility in certain embodiments, such as, e.g., in antibody generation.

1. Elicitation of Immune Response

It is contemplated by the inventors that the proteins, peptides or polypeptides derived from the instant invention may be useful in the elicitation of an immune response. The proteins, peptides or polypeptides may be useful not only in inducing immunity but also in the further derivation of immunogenicity or antigenicity of specific proteins, peptides or polypeptides. Further, the proteins, peptides or polypeptides may be useful in the derivation of specific epitopes as well as the creation of antibodies, including monoclonals and polyclonals.

It is contemplated that expression vectors may be introduced into host organisms according to the ELI protocol set forth in U.S. Pat. Nos. 5,989,553 and 5,703,057. It is contemplated that the ORFs derived from the instant invention will be useful in constructing these expression vectors. In certain embodiments, the present invention therefore provides for a means of eliciting an immune response in a subject. An immune response may be detected in a number of ways. A common manner is to assay for antibody production, however, it is also contemplated that cellular response may be assayed in order to determine immunogenicity. In addition, one can also use an animal challenge model to test for protection against a given pathogen after the vaccination regimen has been administered (U.S. Pat. Nos. 5,989,553 and 5,703,057).

An antibody response may be detected in a number of ways well known in the art. Assays of antibody titer or specificity include: RIA, EIA, ELISA, ELISPOT, western blotting and immunoprecipitation.

Cellular responses may also be used to gauge the nature of the immunogenicity of a peptide, protein or polypeptide. Cellular responses may be measured through techniques well known in the art, including for example: proliferation assays, cytokine assays or cytotoxicity assays.

a. Epitopic Core Sequences

In another aspect, the invention provides a peptide protein or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., 1984.

The proteins, peptides or polypeptides of the invention may further comprise CTL epitopes. CTL epitopes are regions of the molecule capable of activating CD8⁺ T lymphocytes when expressed on the surface of an antigen-presenting cell in the context of MHC class I.

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., 1984. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective. Sutcliffe et al., supra, at 661. For instance, 18 of 20 peptides designed according to these guidelines, containing 8-39 residues covering 75% of the sequence of the influenza virus hemagglutinin HA1 polypeptide chain, induced antibodies that reacted with the HA1 protein or intact virus; and 12/12 peptides from the MuLV polymerase and 18/18 from the rabies glycoprotein induced antibodies that precipitated the respective proteins.

U.S. Pat. No. 4,554,101, (Hopp) incorporated herein by reference, teaches the identification and/or preparation of epitopes from primary amino acid sequences on the basis of hydrophilicity. Through the methods disclosed in Hopp, one of skill in the art would be able to identify epitopes from within an amino acid sequence.

Numerous scientific publications have also been devoted to the prediction of secondary structure, and/or to the identification of epitopes, from analyses of amino acid sequences (Chou and/or Fasman, 1974a,b; 1978a,b, 1979). Any of these may be used, if desired, to supplement the teachings of Hopp in U.S. Pat. No. 4,554,101.

Moreover, computer programs are currently available to assist with predicting antigenic portions and/or epitopic core regions of proteins. Examples include those programs based upon the Jameson-Wolf analysis (Jameson and/or Wolf, 1988; Wolf et al., 1988), the program PepPlot® (Brutlag et al., 1990; Weinberger et al., 1985), and/or other new programs for protein tertiary structure prediction (Fetrow and/or Bryant, 1993). Another commercially available software program capable of carrying out such analyses is MacVector (IBI, New Haven, Conn.).

Antigenic epitope-bearing peptides and polypeptides of the invention are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody reactive with the native protein. Sutcliffe et al., supra, at 663.

Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

Immunogenic epitope-bearing peptides of the invention, i.e., those parts of a protein that elicit an antibody response when the whole protein is the immunogen, are identified according to methods known in the art. For instance, Geysen et al., 1984, supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 to Geysen (1987) further describes this method of identifying a peptide bearing an immunogenic epitope of a desired protein.

Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear C.sub.1-C.sub.7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

In further embodiments, major antigenic determinants of a polypeptide may be identified by an empirical approach in which portions of the gene encoding the polypeptide are expressed in a recombinant host, and/or the resulting proteins tested for their ability to elicit an immune response. For example, PCR™ can be used to prepare a range of peptides lacking successively longer fragments of the C-terminus of the protein. The immunoactivity of each of these peptides is determined to identify those fragments and/or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and/or deprotection, is screened using a polyclonal and/or monoclonal antibody. The antigenic determinants of the peptides which are initially identified can be further localized by performing subsequent syntheses of smaller peptides with larger overlaps, and/or by eventually replacing individual amino acids at each position along the immunoreactive peptide.

Once one and/or more such analyses are completed, polypeptides are prepared that remove and/or add at least the essential features of one and/or more antigenic determinants. The peptides are then employed in the methods of the invention to reduce and/or enhance the production of antibodies when isolated protein and/or gene constructs made by the methods of the present invention is administered to a mammal, preferably a human. Minigenes and/or gene fusions encoding these determinants can also be constructed and/or inserted into expression vectors by standard methods, for example, using PCR™ cloning methodology.

b. Antibody Generation

In certain embodiments, the present invention provides for the creation of antibodies that bind with high specificity to the proteins, peptides or polypeptides produced by the instant invention. As detailed above, in addition to antibodies generated against the full length proteins, antibodies may also be generated in response to smaller constructs comprising epitopic core regions, including wildtype and/or mutant epitopes.

As used herein, the term “antibody” is intended to refer broadly to any immunologic binding agent such as IgG, IgM, IgA, IgD and/or IgE. Generally, IgG and/or IgM are preferred because they are the most common antibodies in the physiological situation and/or because they are most easily made in a laboratory setting.

Once an immune response is elicited in a subject organism by the introduction of the proteins, peptides or polypeptides derived from the instant invention, it is contemplated that antibodies may be isolated which are specific for those proteins, peptides or polypeptides. Monoclonal antibodies (MAbs) are recognized to have certain advantages, e.g., reproducibility and/or large-scale production, and/or their use is generally preferred. The invention thus provides monoclonal antibodies of human, murine, monkey, rat, hamster, rabbit and/or even chicken origin. Due to the ease of preparation and/or ready availability of reagents, murine monoclonal antibodies will often be preferred.

However, “humanized” antibodies are also contemplated, as are chimeric antibodies from mouse, rat, and/or other species, bearing human constant and/or variable region domains, bispecific antibodies, recombinant and/or engineered antibodies and/or fragments thereof. See U.S. Pat. No. 5,482,856. Methods for the development of antibodies that are “custom-tailored to the patient's disease are likewise known and/or such custom-tailored antibodies are also contemplated. For example, humanized antibodies against a specific pathogen can be generated within the scope of the present invention.

The term “antibody” is used to refer to any antibody-like molecule that has an antigen binding region, and/or includes antibody fragments such as Fab′, Fab, F(ab′)₂, single domain antibodies (DABs), Fv, scFv (single chain Fv), and/or the like. The techniques for preparing and/or using various antibody-based constructs and/or fragments are well known in the art. Means for preparing and/or characterizing antibodies are also well known in the art (See, e.g., Antibodies: A Laboratory Manual, Cold Spring Harbor Laboratory, 1988; incorporated herein by reference).

The methods for generating monoclonal antibodies (MAbs) generally begin along the same lines as those for preparing polyclonal antibodies. Briefly, a polyclonal antibody is prepared by immunizing an animal with proteins, peptides or polypeptides in accordance with the present invention and/or collecting antisera from that immunized animal.

A wide range of animal species can be used for the production of antisera. Typically the animal used for production of antisera is a rabbit, a mouse, a rat, a hamster, a guinea pig and/or a goat. Because of the relatively large blood volume of rabbits, a rabbit is a preferred choice for production of polyclonal antibodies. See generally, Stills, 1994.

As is well known in the art, a given composition may vary in its immunogenicity. It is often necessary therefore to boost the host immune system, as may be achieved by coupling a peptide and/or polypeptide immunogen to a carrier. Exemplary and/or preferred carriers are keyhole limpet hemocyanin (KLH) and/or bovine serum albumin (BSA). Other albumins such as ovalbumin, mouse serum albumin and/or rabbit serum albumin can also be used as carriers. Means for conjugating a polypeptide to a carrier protein are well known in the art and/or include glutaraldehyde, m-maleimidobenzoyl-N-hydroxysuccinimide ester, carbodiimide and/or bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins and/or synthetic compositions.

Adjuvants that may be used include IL-1, IL-2, IL-4, IL-7, IL-12, γ-interferon, GMCSP, BCG, aluminum hydroxide, MDP compounds, such as thur-MDP and/or nor-MDP, CGP (MTP-PE), lipid A, and/or monophosphoryl lipid A (MPL). RIBI, which contains three components extracted from bacteria, MPL, trehalose dimycolate (TDM) and/or cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), algammulin incomplete Freund's adjuvants, Gerbu Adjuvant, nitrocellulose adsorbed protein, Montamide ISA, Hunter'TiterMax and/or aluminum hydroxide adjuvant. See, generally Bennett et al., 1992.

In addition to adjuvants, it may be desirable to coadminister biologic response modifiers (BRM), which have been shown to upregulate T cell immunity and/or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, N.J.), cytokines such as γ-interferon, IL-2, and/or IL-12 and/or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and/or intraperitoneal). The production of polyclonal antibodies may be monitored by sampling blood of the immunized animal at various points following immunization.

A second, booster injection, may also be given. The process of boosting and/or titering is repeated until a suitable titer is achieved. When a desired level of immunogenicity is obtained, the immunized animal can be bled and/or the serum isolated and/or stored, and/or the animal can be used to generate MAbs.

For production of rabbit polyclonal antibodies, the animal can be bled through an ear vein and/or alternatively by cardiac puncture. The removed blood is allowed to coagulate and/or then centrifuged to separate serum components from whole cells and/or blood clots. The serum may be used as is for various applications and/or else the desired antibody fraction may be purified by well-known methods, such as affinity chromatography using another antibody, a peptide bound to a solid matrix, and/or by using, e.g., protein A and/or protein G chromatography.

MAbs may be readily prepared through use of well-known techniques, such as those exemplified in U.S. Pat. No. 4,196,265, incorporated herein by reference, see also Antibodies, A Laboratory Manual, Harlow, 1988. Typically, this technique involves immunizing a suitable animal with a selected immunogen composition, e.g., a purified and/or partially purified [GENE 1] and/or [GENE 2] protein, polypeptide, peptide and/or domain, be it a wild-type and/or mutant composition. The immunizing composition is administered in a manner effective to stimulate the production of antibody by B cells.

The methods for generating monoclonal antibodies (MAbs) generally begin along the same lines as those for preparing polyclonal antibodies. Rodents such as mice and/or rats are preferred animals, however, the use of rabbit, sheep and/or frog cells is also possible. The use of rats may provide certain advantages (Goding, 1986, pp. 60-61), but mice are preferred, with the BALB/c mouse being most preferred as this is most routinely used and/or generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete and/or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils and/or lymph nodes, and/or from a peripheral blood sample. Spleen cells and/or peripheral blood cells are preferred, the former because they are a rich source of antibody producing cells that are in the dividing plasmablast stage, and/or the latter because peripheral blood is easily accessible.

Often, a panel of animals will have been immunized and/or the spleen of an animal with the highest antibody titer will be removed and/or the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and/or enzyme deficiencies that render then incapable of growing in certain selective media which support the growth of only the desired fused cells (hybridomas). Other techniques for producing and maintaining antibody secreting lymphocyte cell lines in culture include viral transfection of the lymphocyte to produce a transformed cell line which will continue to grow in culture. Epstein bar virus (EBV) has been used for this technique. EBV-transformed cells do not require fusion with a myeloma cell to allow continued growth in culture.

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and/or S194/5XX0 Bul; for rats, one may use R210.RCY3, Y3-Ag 1.2.3, IR983F and/or 4B210; and/or U-266, GM1500-GRG2, LICR-LON-HMy2 and/or UC729-6 are all useful in connection with human cell fusions.

One preferred murine myeloma cell is the NS-1 myeloma cell line (also termed P3-NS-1-Ag4-1), which is readily available from the NIGMS human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen and/or lymph node cells and/or myeloma cells usually comprise mixing somatic cells with myeloma cells in a 2:1 proportion, though the proportion may vary from about 20:1 to about 1:1, respectively, in the presence of an agent and/or agents (chemical and/or electrical) that promote the fusion of cell membranes. Fusion methods using Sendai virus have been described by Kohler and/or Milstein (1975; 1976), and/or those using polyethylene glycol (PEG), such as 37% (v/v) PEG, by Gefter et al. (1977). The use of electrically induced fusion methods is also appropriate (Goding pp. 71-74, 1986). Where aminopterin and/or methotrexate is used, the media is supplemented with hypoxanthine and/or thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and/or they cannot survive. The B cells can operate this pathway, but they have a limited life span in culture and/or generally die within about two weeks. Therefore, the only cells that can survive in the selective media are those hybrids formed from myeloma and/or B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and/or rapid, such as radioimmunoassays, enzyme immunoassays, cytotoxicity assays, plaque assays, dot immunobinding assays, and/or the like.

The selected hybridomas would then be serially diluted and/or cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and/or myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum and/or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further concentrated and purified, if desired, using precipitation, filtration, and centrifugation and/or various chromatographic methods such as HPLC and/or affinity chromatography (U.S. Pat. No. 5,429,746). Antibody may be precipitated from preparations using techniques which include precipitants such as ammonium sulfate, caprylic acid, DEAE or hydroxyapatite. Techniques combining precipitation with ammonium sulfate and either DEAE or caprylic acid yield nearly pure preparations of antibody. For highly purified preparations, chromatographic techniques employing protein A beads, antigen affinity columns, or anti-Ig affinity columns are preferred.

Fragments of the monoclonal antibodies of the invention can be obtained from the monoclonal antibodies so produced by methods, which include digestion with enzymes, such as pepsin and/or papain, and/or by cleavage of disulfide bonds by chemical reduction. Alternatively, monoclonal antibody fragments encompassed by the present invention can be synthesized using an automated peptide synthesizer or by expression in recombinant systems. See Carter, U.S. Pat. No. 5,648,237.

It is also contemplated that a molecular cloning approach may be used to generate monoclonals. For this, combinatorial immunoglobulin phagemid libraries are prepared from RNA isolated from the spleen of the immunized animal, and/or phagemids expressing appropriate antibodies are selected by panning using cells expressing the antigen and/or control cells. The advantages of this approach over conventional hybridoma techniques are that approximately 10⁴ times as many antibodies can be produced and/or screened in a single round, and/or that new specificities are generated by H and/or L chain combination which further increases the chance of finding appropriate antibodies.

Epitope-bearing peptides and polypeptides of the invention are used to induce antibodies according to methods well known in the art. See, for instance, Sutcliffe et al., supra; Wilson et al., supra; Chow et al., 1985; Bittle et al., 1985. Generally, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine may be coupled to carrier using a linker such as m-maleimidobenzoyl-N-hydroxysuccinimide ester (MBS), while other peptides may be coupled to carrier using a more general linking agent such as glutaraldehyde. Animals such as rabbits, rats and mice are immunized with either free or carrier-coupled peptides, for instance, by intraperitoneal and/or intradermal injection of emulsions containing about 100 μg peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

In another aspect, the invention provides a peptide or polypeptide comprising an epitope-bearing portion of a polypeptide of the invention. The epitope of this polypeptide portion is an immunogenic or antigenic epitope of a polypeptide. An “immunogenic epitope” is defined as a part of a protein that elicits an immune response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., 1984.

Antigenic epitope-bearing peptides and polypeptides of the invention are therefore useful to raise antibodies and generally to induce immunity. Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

The epitope-bearing peptides and polypeptides may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies.

Immunogenic epitope-bearing peptides of the invention, i.e., those parts of a protein that elicit an immune response when the whole protein is the immunogen, are identified according to methods known in the art. For instance, Geysen et al., 1984, supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 1987 further describes this method of identifying a peptide bearing an immunogenic epitope of a desired protein.

Once one and/or more such analyses are completed, polypeptides are prepared that remove and/or add at least the essential features of one and/or more antigenic determinants. The peptides are then employed in the methods of the invention to reduce and/or enhance the production of antibodies when isolated protein and/or gene constructs made by the methods of the present invention is administered to a mammal, preferably a human. Minigenes and/or gene fusions encoding these determinants can also be constructed and/or inserted into expression vectors by standard methods, for example, using PCR™ cloning methodology.

c. Serological Assays

The present invention includes detecting an immune response These assays take advantage of antigen-antibody interactions to quantify and qualify antigen levels. There are many types of assays that can be implemented, some of which are presented herein, which one of ordinary skill in the art would know how to implement in the scope of the present invention.

i. Immunoassay and Immunohistological Assays

Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful.

In one exemplary ELISA, the antibodies are immobilized on a selected surface, such as a well in a polystyrene microtiter plate, dipstick, or column support. Then, a test composition suspected of containing the desired antigen, such as a clinical sample, is added to the wells. After binding and washing to remove non-specifically bound immune complexes, the bound antigen may be detected. Detection is generally achieved by the addition of another antibody, specific for the desired antigen, that is linked to a detectable label. This type of ELISA is known as a “sandwich ELISA”. Detection also may be achieved by the addition of a second antibody specific for the desired antigen, followed by the addition of a third antibody that has binding affinity for the second antibody, with the third antibody being linked to a detectable label.

Variations on ELISA techniques are known to those of skill in the art. In one such variation, the samples suspected of containing the desired antigen are immobilized onto the well surface and then contacted with the antibodies of the invention. After binding and appropriate washing, the bound immune complexes are detected. Where the initial antigen specific antibodies are linked to a detectable label, the immune complexes may be detected directly. Again, the immune complexes may be detected using a second antibody that has binding affinity for the first antigen specific antibody, with the second antibody being linked to a detectable label.

Competition ELISAs are also possible in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non-specifically bound species, and detecting the bound immune complexes. These are described as below.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

In ELISAs, it is more customary to use a secondary or tertiary detection means rather than a direct procedure. Thus, after binding of the antigen or antibody to the well, coating with a non-reactive material to reduce background, and washing to remove unbound material, the immobilizing surface is contacted with the clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The suitable conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C. or so.

After all incubation steps in an ELISA are followed, the contacted surface is washed so as to remove non-complexed material. Washing often includes washing with a solution of PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase, or hydrogen peroxidase-conjugated antibody for a period of time and under conditions that favor the development of further immune complex formation, e.g., incubation for 2 hours at room temperature in a PBS-containing solution such as PBS-Tween.

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

Alternatively, the label may be a chemiluminescent one. The use of such labels is described in U.S. Pat. Nos. 5,310,687, 5,238,808 and 5,221,605.

Assays for the presence of an HLA haplotype may be performed directly on tissue samples. Methods for in vitro situ analysis are well known and involve assessing binding of antigen-specific antibodies to tissues, cells, or cell extracts. These are conventional techniques well within the grasp of those skilled in the art.

J. Immunity and Pathogenicity

It is contemplated that the composition of the instant invention may be used in the determination of immunogenic or antigenic proteins, polypeptides, peptides or more specifically immunogenic epitopes of specific pathogens. These peptides are envisioned to be useful in the elicitation of an immune response in a host organism. A purpose of the invention is thus, ultimately to isolate a protein or peptide capable of eliciting a partial or fully protective immune response in a host. For the purpose of the invention, the type of immune response envisioned may be of a cellular and/or humoral nature. A cellular or delayed type hypersensitivity response involves the induction of specific cellular components of the immune system to eliminate a pathogen from the host. In contrast, humoral immunity is based upon the ability of an antigen to induce B-cells to produce antibody.

Adaptive immunity or memory is directed against specific molecules and is enhanced by re-exposure. Adaptive immunity is mediated by cells called lymphocytes, which synthesize cell-surface receptors, secrete signaling molecules or secrete proteins that bind specifically to foreign molecules. A subset of these secreted proteins are known as antibodies. Any molecule that can bind to an antibody is known as an antigen. Antigenicity also is not an intrinsic property of a molecule, but is defined by its ability to be bound by an antibody.

The term “immunoglobulin” is often used interchangeably with “antibody.” Formally, an antibody is a molecule that binds to a known antigen, while immunoglobulin refers to this group of proteins irrespective of whether or not their binding target is known. This distinction is trivial and the terms are used interchangeably.

Many types of lymphocytes with different functions have been identified. Most of the cellular functions of the immune system can be described by grouping lymphocytes into three basic types—B cells, cytotoxic T cells, and helper T cells. All three carry cell-surface receptors that can bind antigens. B cells secrete antibodies, and carry a modified form of the same antibody on their surface, where it acts as a receptor for antigens. Cytotoxic T cells lyse foreign or infected cells, and they bind to these target cells through their surface antigen receptor, known as the T-cell receptor. Helper T cells play a key regulatory role in controlling the response of B cells and cytotoxic T cells, and they also have T-cell receptors on their surface.

T-cell activation is an important step in the protective immunity against pathogenic microorganisms (e.g., viruses, bacteria, and parasites) and foreign proteins, and particularly those that reside inside affected cells. T cells express receptors on their surface (i.e., T-cell receptors), which recognize antigens presented on the surface of antigen-presenting cells. During a normal immune response, binding of these antigens to the T cell receptor initiates intracellular changes leading to T-cell activation. T cells are divided into specific subsets that are generally defined by antigenic determinants found on their cell surfaces, as well as functional activity and foreign antigen recognition. CD4 lymphocytes generally include the T-helper and T-delayed type hypersensitivity subsets. The CD4 protein typically interacts with Class II major histocompatibility complex. CD4 may function to increase the avidity between the T cell and its MHC class II APC or stimulator cell and enhance T cell proliferation. CD8 lymphocytes are generally cytotoxic T-cells, whose function is to identify and kill foreign cells or host cells displaying foreign antigens. The CD8 protein typically interacts with Class I major histocompatibility complex.

One of the key features of the immune system is that it can synthesize a vast repertoire of antibodies and cell-surface receptors, each with a different antigen binding site. The binding of the antibodies provides the molecular basis for the specificity of a humoral immune response. B cells are defined by their ability to differentiate into cells capable of secreting antibody. Mature B cells surface express antibody with a unique antigen specificity. In response to the crosslinking of surface antibody and with the aid of helper T cells, B cells differentiate into plasma cells capable of secreting soluble antibody.

The specificity of the immune response is controlled by a simple mechanism—one cell recognizes one antigen because all of the antigen receptors on a single lymphocyte are identical. This is true for both T and B lymphocytes, even though the types of responses made by these cells are different.

All antigen receptors are glycoproteins found on the surface of mature lymphocytes. Somatic recombination, mutation, and other mechanisms generate more than 10⁷ different binding sites, and antigen specificity is maintained by processes that ensure that only one type of receptor is synthesized within any one cell. The production of antigen receptors occurs in the absence of antigen. Therefore, a diverse repertoire of antigen receptors is available before antigen is seen.

Although they share similar structural features, the surface antibodies on B cells and the T-cell receptors found on T cells are encoded by separate gene families; their expression is cell-type specific. The surface antibodies on B cells can bind to soluble antigens, while the T-cell receptors recognize antigens only when displayed on the surface of other cells.

When B-cell surface antibodies bind antigen, the B lymphocyte is activated to secrete antibody and is stimulated to proliferate. T cells respond in a similar fashion. This burst of cell division increases the number of antigen-specific lymphocytes, and this clonal expansion is the first step in the development of an effective immune response. As long as the antigen persists, the activation of lymphocytes continues, thus increasing the strength of the immune response. After the antigen has been eliminated, some cells from the expanded pools of antigen-specific lymphocytes remain in circulation. These cells are primed to respond to any subsequent exposure to the same antigen, providing the cellular basis for immunological memory.

In the first step in mounting an immune response the antigen is engulfed by an antigen presenting cell (APC). The APC degrades the antigen and pieces of the antigen are presented on the cell surface by a glycoprotein known as the major histocompatibility complex class II proteins (MHC II). Helper T-cells bind to the APC by recognizing the antigen and the class II protein. The protein on the T-cell which is responsible for recognizing the antigen and the class II protein is the T-cell receptor (TCR).

Once the T-cell binds to the APC, in response to Interleukin 1 and 2 (IL), helper T-cells proliferate exponentially. In a similar mechanism, B cells respond to an antigen and proliferate in the immune response. The ability of a clonal population of immune cells to expand in response to a determinative antigen allows for the immune system to expand the population best suite to respond to a specific infectious agent or pathogen.

The term pathogen is defined for the purpose of the invention as an element capable of inducing disease in a host organism. A pathogen is more specifically considered to encompass any prion, virion, viroid, virus, bacteria, rickettsial, fungus, protozoan, algae, plant, helminth, or other metazoan capable of causing a disease. Specific organisms contemplated by the inventors to be a particular focus of the invention are those organisms capable of antigenic shift, antigenic drift or molecular mimicry. Such organisms include, but are not limited to: Trypanosoma brucei, Plasmodia falciporum, Schistosoma mansoni, Entamoeba hystilytica, and Toxoplasma gondii.

K. Pharmaceutical Compositions

It is contemplated that products of the methods and compositions of the claimed invention may be delivered into a host organism.

1. Pharmaceutically Acceptable Carriers

In some embodiments of the present invention expression constructs are given to an animal potentially to elicit an immune response in the animal. An immune response could lead to the identification of antigenic determinants encoded by the expression construct, for example. Thus, aqueous compositions of expression constructs expressing any of the foregoing are also contemplated. Similarly genomic immunization employs the delivery of a nucleic acid vector that delivers a DNA-encoded sequence for vaccination purposes. These vectors, in addition to the proteins, peptides or polypeptides derived from the composition of the invention may be dissolved or dispersed in a pharmaceutically acceptable carrier or aqueous medium for delivery to a host organism. See Sykes et al., 1999, herein specifically incorporated by reference. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an expression construct (viral vectors included) and/or antibodies as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The composition may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

2. Liposomes and Nanocapsules

In certain embodiments, the use of liposomes and/or nanoparticles is contemplated for the introduction of formulations of expression constructs, proteins, peptides or polypeptides of the invention. The formation and use of liposomes is generally known to those of skill in the art, and is also described below.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

I. Kits

The materials and reagents required for detecting open reading frames in a biological sample may be assembled together in a kit. The kits of the invention generally will comprise a ORF-selection vector. In some embodiments, an expression construct that can be used after an ORF has been identified to practice the method of, for example, ELI, may be included. Other components of kits of the present invention may include one or more of the following: a set of restriction endonucleases used to digest the nucleic acids, ligase, phosphatase, and any other useful agent for the use and practice of the claimed compositions and methods.

In each case, the kits will preferably comprise distinct containers for each individual component. Each biological agent will generally be suitable aliquoted in their respective containers. The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Construction of pORF-GFP

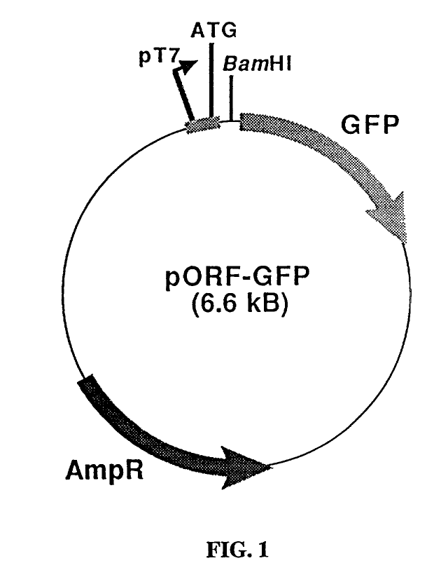

The open reading frame selection vector pORF-GFP was derived from plasmid pCMViUB (Sykes and Johnston, 1999). Briefly, the GFP gene from pBAD-GFP (Crameri et al., 1996) was inserted into the cloning region of pCMViUB. The bacteriophage T7 promoter and cognate Shine-Dalgamo region of pET-3a (Studier et al., 1990) was cloned upstream of the GFP gene, with the initiating ATG positioned out of frame with respect to the GFP reading frame. In addition, the termination sequence of T7 from plasmid pET-3 (Studier et al., 1990) was cloned downstream of the GFP reporter gene. A unique BamHI site was placed between the initiating ATG and the start of the GFP gene to produce parent plasmid pORF-GFP, which is shown in FIG. 1a. To produce plasmid pORF-PBA-GFP, unique restriction sites for PacI and AscI were inserted on either side of the BamHI site (FIG. 1b). In addition, the region immediately upstream of the GFP gene was replaced with an alanine-rich linker, and the initiation ATG codon of GFP was replaced with a GCG codon for alanine. Plasmid pORF-PNA-GFP was derived from pORF-PBA-GFP by the replacement of the BamHI restriction site with a NarI site (FIG. 1c).

Cloning of Genomic DNA and Selection of ORF-GFP Fusions

Vector DNA was prepared by digesting the pORF-GFP plasmid with BamHI and treating with calf alkaline phosphatase (Promega, Madison, Wis.) according to the manufacturer's specifications. Genomic DNA from Saccharomyces cerevisiae was prepared using standard techniques described in Sambrook et al.,(1989). Insert DNA was prepared by partial digestion with Sau3A, followed by size-fractionation on a 1% agarose gel and purification on a Qiaquick gel extraction column (Qiagen). Insert DNA was cloned using standard ligation conditions and transformed into E. coli host strain HMS174(DE3) (Novagen) by electroporation (Biorad). Transformants were spread onto LB agar plates supplemented with ampicillin (75 μg/ml), chloramphenicol (20 μg/ml) and IPTG (40 μM), and grown at 30° C. for 40 to 48 hr, at which time GFP expression was readily apparent upon irradiation with a standard long-wavelength UV light source.

Insert Analysis

Plasmid DNA was isolated from clones using the Wizard Kit (Promega, Madison, Wis.). Inserts were sequenced using the BigDye Terminator Cycle Sequencing Ready Reaction kit from PE Applied Biosystems (Foster City, Calif.) and analyzed on an ABI automated sequencer. The forward primer 5′ CCCTGACCGGCAAGACCA 3′ and/or reverse primer 5′ TTGGACAACTCCAGTGAAAA 3′ were used for sequencing of inserts. Homology searches were carried out using the BLAST program to search the Saccharomyces genome database (http://genome-www.stanford.edu/Saccharomyces). Statistical analysis of ORF frequency was achieved as follows: the GORF/STORF distributions were generated from annotated and raw sequence obtained from the NCBI website (www.ncbi.nlm.nih.gov). For Plasmodium falciparum the coding sequences for chromosomes 2 & 3 were extracted from the Genbank files using parsing engines and were then combined to generate a single GORF distribution. The STORF distribution was generated by identifying all sequences between adjacent stop codons in all six reading frames for both chromosomes and subtracting out of GORF distributions. These were also combined into a single STORF distribution.

Construction of a Selection Vector for Open Reading Frames

The GFP gene was chosen as the reporter in our open reading frame selection vector on the basis of several criteria: 1) In contrast to other reporter genes that are typically based on enzymatic activities, GFP encodes a non-enzymatic function which is less likely to be adversely affected by fusions; 2) GFP is an unusually stable protein which renders it resistant to most proteases for many hours, and its spectral properties are unaffected when denatured; 3) GFP expression can be detected on irradiation using a standard UV light source without the introduction of a substrate; 4) the relatively small size of GFP (238 amino acids) and monomeric nature facilitate the formation of stable protein fusions (Prasher, 1995; Cubitt et al., 1995; Tsien, 1998). To increase the number of stable GFP fusions which can be detected with pORF-GFP, the inventors incorporated a synthetic version of GFP with improved codon usage for E. coli expression systems as well as increased solubility and fluorescence relative to wild type GFP (Crameri et al., 1996). Furthermore, it has been observed that proteins fused with this synthetic GFP can maintain fluorescence even when they are insoluble and trapped within inclusion bodies (Russell and Johnston, unpublished results). Consequently, the ORF-encoded portion of a GFP fusion protein does not need to be in a functional state in the initial screen.

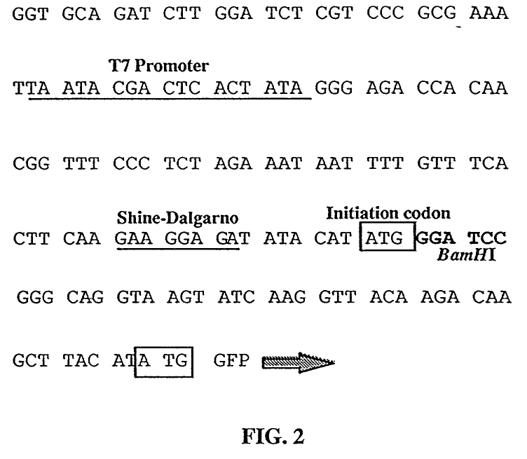

The pORF-GFP vector contains a bacteriophage T7 transcription/translation sequence, with the initiating ATG codon being out of frame with the GFP reporter gene (FIG. 2a). Insertion of DNA fragments with a length of 3n+1 between these two sequences is required to allow translation of an ORF-GFP fusion. The presence of the T7 promoter allows high levels of expression to occur upon IPTG induction; conversely expression can be minimized during subsequent amplification steps by omission of IPTG to preclude possible mutation and/or loss of plasmid clones. To confirm that the pORF-GFP vector could indeed provide a distinguishable phenotype, a thymine residue was inserted upstream of the GFP gene to bring it in frame with the initiating ATG. Colonies of E. coli that contained this construct fluoresced strongly when grown in the presence of IPTG, whereas those containing the pORF-GFP vector were white. Furthermore, no leakage of expression of GFP from the vector was observed at any stage.

Testing the pORF-GFP Selection Vector with Saccharomyces cerevisiae Genomic DNA

To accurately determine the efficacy of pORF-GFP as an open reading frame selection vector, genomic DNA libraries were prepared from Saccharomyces cerevisiae. Genomic libraries containing size-selected Sau3A-partially digested S. cerevisiae DNA were constructed by cloning into the BamHI site of pORF-GFP, and transformants were screened for green fluorescence. In preliminary studies, it was found that the growth conditions during IPTG induction affected the number of false positives (namely, those fluorescent colonies that contained non-ORF inserts); this number could be reduced by 1) lowering the IPTG concentration from the standard 100 μM to 40 μM and 2) incubating the plated bacteria at 30° C. Using these optimized conditions, four independent genomic libraries were screened for ORFs, and the results of the observed phenotypes are summarized in Table 6. Of the total 3120 colonies screened, 129 colonies (4%) had a green fluorescent phenotype, consistent with the production of functional ORF-GFP fusion proteins. Given that approximately 80% of the S. cerevisiae genome is predicted to encode genes (Mewes et al., 1997), the observed frequency of ORF-containing colonies is consistent with the predicted frequency of 4.4% (1/18×⅘). The intensity of fluorescence varied between the colonies, and allowed the putative ORF-containing green colonies to be arbitrarily classified as bright, medium or pale green, and the relative frequencies are shown in Table 6.

TABLE 6

| Total | Number of | Number of | Number | Number of |
|---|---|---|---|---|
| number | green | clones | of | authetic |
| of colonies | colonies | sequence | ORFs | genes |
| 3120 | 129 (4.1%) | 90 | 49 (54%) | 22 (24%) |

In order to measure the efficacy of the ORF screen and to determine whether there was a relationship between insert identity and intensity of fluorescence, the cloned inserts from 90 green colonies were sequenced (Table 7). Of the 90 selected clones, 26, 35 and 29 had bright, medium or pale green phenotypes, respectively. Analysis of these sequenced inserts showed that 49 out of 90 (54%) were ORFs based on the criteria that 1) they that linked the initiating ATG codon of pORF-GFP in frame with the GFP reporter gene and 2) they contained no stop codons. The frequency of ORFs by these criteria was found to be greatest for the most fluorescent colonies, with 85% of bright green colonies containing ORFs in contrast to only 43% and 41% of medium and pale green colonies containing ORFs, respectively. Upon closer inspection, a pronounced inverse relationship between insert length and intensity of fluorescence was observed, with bright, medium and pale green colonies carrying inserts with respective average lengths of 208 bp, 336 bp and 529 bp. It was also observed that larger non-ORF inserts were more likely to give rise to false positives as a consequence of an increased probability of containing an internal promoter and/or Shine-Dalgarno sequence that allows GFP expression to occur.

TABLE 7

| | Total number | Number of | | Number |
|---|---|---|---|---|
| | of | green | Number | of |
| Parasite | colonies | colonies | sequenced | ORFs |
| N. caninum | 330 | 32 (10%) | 32 | 22 (85%) |
| T. cruzi | 409 | 8 (2%) | 6 | 5 (83%) |

To determine whether the 49 ORFs identified using pORF-GFP corresponded to the ORFs of predicted genes, the translated genomic database of S. cerevisiae was searched with each of the translated ORF sequences. Interestingly, the inventors found that 80% (12/15) of ORFs from medium fluorescent colonies corresponded to real genes, whereas ORFs from pale green colonies had a correspondence of 50% (6/12). By contrast, only 18% (4/22) of the ORFs from bright green colonies were genes, indicating that a large proportion of the inserts within these clones are likely to contain fortuitous ORFs (those in frame and without stop codons) by virtue of their small size. In total, 22 of the 49 ORFs (54%) identified in this screen corresponded to genes. This proportion can be increased by more stringent selection of the insert size range to eliminate fortuitous ORFs.

To ascertain whether there was any bias in the cloning or selection of gene ORFs, the identity (and function, where known) of each sequence was determined from the yeast genome database. Of the 22 ORFs that were identified as genes, 17 were unique clones, while 2 independent clones appeared to map to the same gene of unknown function. Curiously, 3 of the gene ORFs corresponded to the 25srRNA gene; however, 7 of the 27 ORFs that were not in frame with the gene also contained 25srRNA sequence. This frequency exceeds the elevated number of such clones which would be anticipated (since the S. cerevisiae genome contains approximately 100 copies of the 25srRNA gene), suggesting that this ribosomal RNA sequence allows spurious translation of GFP to occur.

Based on the results of the S. cerevisiae-pORF-GFPS test library, a number of chnages were incorporated into the vector to optimize its selectivity and versatility for genomic screening. The ATG start codon of the GFP gene was deleted to reduce the incidence of spurious readthrough from Shine Dalgamo-like sequences within the insert. To increase the stability of GFP fusions proteins, the sequence immediately upstrweam of the GFP gene was modified to encode an alanine-rich linker. For subsequent excision of DNA inserts, sites for restriction enzymes PacI and AscI (which recognize 8 bp sequences) were introduced to span the BamHI site in such a way as to maintain the orientation and reading frame of the inserts upon subcloning. The resultant vector pORF-PBA-GFP is shown in FIG. 2b. To increase the cloning flexibility of the system, the BamHI site of pORF-PBA-GFP was replaced with a site for NarI (which is compatible with the enzymes TaqI, MaeII, MspI, AciI and HinP1I), resulting in vector pORF-PCA-GFP (FIG. 2c).

Testing the pORF-GFP Vector with Eukaryotic Parasite DNA

To test the efficacy of pORF-GFP for selecting ORFs from complex genomic DNA, genomic libraries were prepared with partially Sau3a-digested DNA from the eukaryotic parasites Neospora caninum and Trypanosoma cruzi. The results of these screens showed that N. caninum and T. cruzi inserts gave rise to green fluorescent colonies at frequencies of 10% and 2%, respectively (Table 8, top panel). Sequence analysis of putative ORFs from positive colonies revealed that approximately 85% of the sequences were indeed ORFs, with most of the false positives attributable to the presence of translation initiation signals within the inserts. To determine if elimination of the initiating ATG codon of GFP would decrease the frequency of false ORFs, genomic libraries of Sau3A-partially digested N. caninum and T. cruzi DNA were prepared in pORF-PBA-GFP. Screening of the libraries (Table 3, lower panel) revealed similar frequencies of fluorescent green colonies as observed with pORF-GFP. In contrast to the parent vector, however, all of the clones prepared in pORF-PBA-GFP corresponded to ORFs, indicating that the latter vector is less likely to give rise to false positives. Finally, sequence analysis of the N. caninum and T. cruzi ORFs identified with vectors pORF-GFP and pORF-PBA-GFP showed that they were all different, indicating that there is no overt bias for the selection of certain gene sequences.

TABLE 8

| | Total | Number of | | |
|---|---|---|---|---|
| | number of | green | Number | Number of |
| Parasite | colonies | colonies | sequenced | ORFs |
| N. caninum | 422 | 36 (9%) | 10 | 10 (100%) |
| T. cruzi | 675 | 26 (4%) | 3 | 3 (100%) |

### EXAMPLE 2

ORF Positive Selection Vector

A plasmid vector (pORF-DD) can be constructed that contains a bacterial “death (toxin) gene” located upstream of a protein “degradation signal”. The death gene and the degradation signal will be out of frame with respect to each other and separated by a cloning cassette. Genomic DNA will be cloned into a site located immediately downstream of the death gene. Consequently, this will result in death of all protein-expressing cells. The C-terminal destruction sequence will be out of frame with respect to the N-terminal death gene, so that only clones that contain ORFs linking the death gene in frame with the destruction sequence will target the “toxin-ORF-destruction signal” protein for proteolysis. Thus, only ORF-containing clones will survive. A number of expression constructs containing different death genes will be constructed and used to identify one that works most effectively in our system. If successful, this strategy can reduce the size of the primary ORF screen (an important consideration for high-throughput screening). Another advantage is that the fusion protein is destroyed, thus removing any deleterious effects due to protein overexpression. This strategy will also benefit from the pORF-GFP data, since optimization of insert size, ligation and transformation should be similar for both systems.

ELI Vector Construction

The pORF-GFP and pORF-DD plasmids are bacterial expression vectors, and thus are less desirable for vaccine screening in animals. To test the selected ORFs in mammalian hosts, a simple strategy will be used of subcloning the ORF-containing fragments into the in-house ELI vectors (which the inventors will modify to allow compatibility with the cloned ORFs). For high-throughput, the genes will be simultaneously subcloned in sets of 96. To initially test whether all 96 fragments are subcloned and no overt biases are generated, a microarray of 96 fluoresecent pORF-GFP clones will be used to probe the subcloned library. The long-term goal is to directly incorporate the features necessary for mammalian expression into pORF-GFP and/or pORF-DD. These include a strong mammalian promoter, and a leader sequence to direct the fusion protein to the appropriate part of the cell to bias the immune system towards a cellular or humoral response. In addition, introns will be incorporated into the vectors to allow splicing of the bacterial selection genes (namely, the GFP and death genes) so that these proteins are not expressed in the mammalian host. For example, an intron will be inserted into a reporter gene. In essence, this will result in one-step ELI-ORF vector.

Thus, this system has uses in the generation of both components involved in an immune response and vaccines depending upon which organism's genome is used in the system.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,196,265

U.S. Pat. No. 4,367,110

U.S. Pat. No. 4,433,092

U.S. Pat. No. 4,452,901

U.S. Pat. No. 4,554,101

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,683,195

U.S. Pat. No. 4,683,202

U.S. Pat. No. 4,708,781

U.S. Pat. No. 4,797,368

U.S. Pat. No. 4,800,159

U.S. Pat. No. 4,883,750

U.S. Pat. No. 5,139,941

U.S. Pat. No. 5,194,392

U.S. Pat. No. 5,196,524

U.S. Pat. No. 5,221,605

U.S. Pat. No. 5,238,808

U.S. Pat. No. 5,296,375

U.S. Pat. No. 5,304,487

U.S. Pat. No. 5,310,687

U.S. Pat. No. 5,429,746

U.S. Pat. No. 5,480,971

U.S. Pat. No. 5,482,856

U.S. Pat. No. 5,491,084

U.S. Pat. No. 5,625,048

U.S. Pat. No. 5,648,237

U.S. Pat. No. 5,703,057

U.S. Pat. No. 5,777,079

U.S. Pat. No. 5,783,402

U.S. Pat. No. 5,804,387

U.S. Pat. No. 5,856,174

U.S. Pat. No. 5,874,304

U.S. Pat. No. 5,904,824

U.S. Pat. No. 5,925,565

U.S. Pat. No. 5,928,906

U.S. Pat. No. 5,935,819

U.S. Pat. No. 5,975,216

U.S. Pat. No. 5,981,177

U.S. Pat. No. 5,981,182

U.S. Pat. No. 5,989,553

EP 0 655 506 B1

EPO 0273085

European Patent Application No. 320,308

European Patent Application No. 329,822

GB 2,202,328

PCT/US87/00880

PCT/US89/01025

WO 88/10315

WO 89/06700

WO 90/07641

WO 94/05414

Agrawal et al., Biology of Antisense RNA and DNA, 273-283, 1992.

Almendro et al., J. Immunol., 157(12):5411-21, 1996.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Ausubel et al., Current Protocols in Molecular Biology John Wiley & Sons, Inc., New York, 1996.

Ausubel, Brent, Kingston, Moore, Seidman, Smith, Struhl (ed.), In: Current protocols in Molecular Biology. John Wiley & Sons, Inc., New York, 1994.

Baichwal and Sugden, In: Gene transfer, Kucherlapati R, ed., New York: Plenum Press, 117-148, 1986.

Banerji et al., Cell, 27:299, 1981.

Banerji et al., Cell, 35:729, 1983.

Barry et al., Nature, 377:632-635, 1995.

Bennett et al., J. of Immunol. Meth., 153:31-40, 1992.

Berkhout et al., Cell, 59:273, 1989.

Blanar et al., EMBO J., 8:1139, 1989.

Bodine and Ley, EMBO J., 6:2997, 1987.

Boshart et al., Cell, 41:521, 1985.

Bosze et al, EMBO J., 5:1615, 1986.

Braddock et al., Cell, 58:269, 1989.

Brutlag et al., CABIOS, 6:237-245, 1990.

Bugge and Gerdes, Mol. Micro., 17:205-210, 1995.

Bulla and Siddiqui, J. Virol., 62:1437, 1986.

Campbell and Villarreal, Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, Genes and Dev., 3:537, 1989.

Campo et al., Nature, 303:77, 1983.

Carbonelli et al., FEMS Microbiol. Lett., 177(1):75-82, 1999.

Celander and Haseltine, J. Virology, 61:269, 1987.

Celander et al., J Virology, 62:1314, 1988.

Chandler et al., Cell, 33:489, 1983. Chang et al., Hepatology, 14:134A, 1991.

Chang et al., Mol. Cell. Biol., 9:2153, 1989.

Chatterjee et al., Proc. Nat'l Acad. Sci. USA., 86:9114, 1989.

Choi et al., Cell, 53:519, 1988.

Chou and Fasman, Biochemistry, 13(2):211-222, 1974b.

Chou and Fasman, Ann. Rev. Biochem., 47:251-276, 1978b.

Chou and Fasman, Biophys. J., 26:367-384, 1979.

Chou and Fasman, Biochemistry, 13(2):222-245, 1974a.

Chou and Fasman, Adv. Enzymol. Relat. Areas Mol. Biol., 47:45-148, 1978a.

Clark et al, Human Gene Therapy, 6:1329-1341, 1995.

Cocea, Biotechniques, 23(5):814-6, 1997.

Coffin, In: Virology, Fields et al. (eds.), New York: Raven Press, pp. 1437-1500, 1990.

Cohen et al., Proc. Nat'l Acad. Sci. USA 75, 472-476, 1978.

Costa et al., Mol. Cell. Biol., 8:81, 1988.

Cotten et al., Proc. Natl. Acad. Sci. USA, 89:6094-6098, 1992. Couch et al., Am. Rev. Resp. Dis., 88:394-403, 1963.

Coupar et al., Gene, 68:1-10, 1988.

Crameri et al., Nature Biotech. 14, 315-319, 1996.

Cripe et al., EMBO J., 6:3745, 1987.

Cubitt et al., TIBS 20:448-455, 1995.

Culotta and Hamer, Mol. Cell. Biol., 9:1376, 1989.

Curiel, In: Viruses in Human Gene Therapy, J.-M. H. Vos (Ed.), Carolina Academic Press, Durham, N.C., 179-212, 1994.

Dandolo et al., J. Virology, 47:55, 1983.

Daugelat and Jacobs, Protein Sci., 8:644-653, 1999.

De Villiers et al., Nature, 312:242, 1984.

Deschamps et al., Science, 230:1174, 1985.

Diaz et al., Mol. Microbiol., 13(5):855-61, 1994.

Edbrooke et al., Mol. Cell. Biol., 9:1908, 1989.

Edlund et al., Science, 230:912, 1985.

Effenhauser et al., Anal. Chem., 65:2637-2642, 1993.

Effenhauser et al., Anal. Chem., 66:2949-2953, 1994.

Fechheimer et al., Proc. Natl. Acad. Sci. USA, 84:8463-8467, 1987

Feng and Holland, Nature, 334:6178, 1988.

Fetrow and Bryant, Biotech., 11:479-483, 1993.

Firak and Subramanian, Mol. Cell. Biol., 6:3667, 1986.

Flotte et al., Am. J. Respir. Cell Mol. Biol., 7:349-356, 1992.

Flotte et al., Gene Therapy, 2:29-37, 1995.

Flotte et al., Proc. Natl. Acad. Sci. USA, 90:10613-10617, 1993.

Fraley and Fomari Kaplan, Proc. Nat'l. Acad. Sci. USA, 76:3348-3352, 1979.

Freifelder and Better, Anal. Biochem., 123(1):83-85, 1982.

Friedmann, Science, 244:1275-1281, 1989.

Frohman, M. A., In “PCR Protocols: A Guide to Methods and Applications,” Academic Press, New York, 1990.

Fujita et al., Cell, 49:357, 1987.

Gefter et al., Somatic Cell Genet. 3:231-236, 1977.

Geysen et al., Proc. Natl. Acad. Sci. USA, 81:3998-4002, 1984.

Gilles et al., Cell, 33:717, 1983.

Gloss et al., EMBO J., 6:3735, 1987.

Godbout et al., Mol. Cell. Biol., 8:1169, 1988.

Goding, In: Monoclonal Antibodies: Principles and Practice, 2d ed., Orlando, Fla., Academic Press, pp. 60-61, 65-66, 71-74, 1986.

Goodbourn and Maniatis, Proc. Nat'l Acad. Sci. USA, 85:1447, 1988.

Goodbourn et al., Cell, 45:601, 1986.

Gopal, Mol. Cell. Biol., 5:1188-1190, 1985.

Gotfredsen and Gerdes, Mol. Micro., 29:1065-1076, 1998.

Gottesman, Curr. Opin. Micro., 2:142-147, 1999.

Graham and Prevec, Biotechnology, 20:363-390, 1992.

Graham and Van Der Eb, Virology, 52:456-467, 1973.

Graham et al., J. Gen. Virol., 36:59-72, 1977.

Greene et al., Immunology Today, 10:272, 1989.

Grosschedl and Baltimore, Cell, 41:885, 1985.

Grunhaus and Horwitz, Seminar in Virology, 3:237-252, 1992.

Gultyaev et al., J. Mol. Biol., 273(1):26-37, 1997.

Harland and Weintraub, J. Cell Biol. 101:1094-1099, 1985.

Harlow and Lane, In: Antibodies: a Laboratory Manual, Cold Spring Harbor Laboratory, Cold Spring Harbor, N.Y., 1988.

Harrison et al., In: Science, 261:895-897, 1993.

Haslinger and Karin, Proc. Nat'l Acad. Sci. USA., 82:8572, 1985.

Hauber and Cullen, J. Virology, 62:673, 1988.

Hen et al., Nature, 321:249, 1986.

Hensel et al., Lymphokine Res., 8:347, 1989.

Hermonat and Muzyczka, Proc. Nat'l. Acad. Sci. USA, 81:6466-6470, 1984.

Herr and Clarke, Cell, 45:461, 1986.

Herz and Gerard, Proc. Nat'l. Acad. Sci. USA, 90:2812-2816, 1993.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Horwich et al. J. Virol., 64:642-650, 1990.

Huang et al., Cell, 27:245, 1981.

Hunkapiller, Curr. Op. Gen. Devl., 1:88-92, 1991.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Jacobsen et al., Anal. Chem., 66:1107-1113, 1994.

Jakobovits et al., Mol. Cell. Biol., 8:2555, 1988.

Jameel and Siddiqui, Mol. Cell. Biol., 6:710, 1986.

Jameson and Wolf, Comput. Appl. Biosci., 4(1):181-186, 1988.

Jaynes et al., Mol. Cell. Biol., 8:62, 1988.

Jensen and Gerdes, Mol. Microbiol., 17(2):205-10, 1995.

Johnson et al., Mol. Cell. Biol., 9:3393, 1989.

Kadesch and Berg, Mol. Cell. Biol., 6:2593, 1986.

Kaneda et al., Science, 243:375-378, 1989.

Kaplitt et al., Nature Genetics, 8:148-154, 1994.

Karin et al., Mol. Cell. Biol., 7:606, 1987.

Kasahara et al., Science, 266:1373-1376, 1994.

Katinka et al., Cell, 20:393, 1980.

Katinka et al., Nature, 290:720, 1981.

Kato et al., J. Biol. Chem., 266:3361-3364, 1991.

Kawamoto et al., Mol. Cell. Biol., 8:267, 1988.

Kelleher and Vos, Biotechniques, 17(6):1110-1117, 1994.

Kiledjian et al., Mol. Cell. Biol., 8:145, 1988.

Klamut et al., Mol. Cell. Biol., 10: 193, 1990.

Klein et al., Nature, 327:70-73, 1987.

Koch et al., Mol. Cell. Biol., 9:303, 1989.

Kohler et al., Nature, 256(5517):495-497, 1975.

Kohler et al., Eur. J. Immunol., 6(7):511-519, 1976.

Kotin et al., Proc. Natl. Acad. Sci. USA, 87:2211-2215, 1990.

Kraus et al., FEBS Lett., 428(3):165-70, 1998.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Kwoh et al., Proc. Natl. Acad. Sci, USA, 86(4):1173-1177, 1989.

LaFace et al., Viology, 162:483-486, 1988.

Lareyre et al., J. Biol. Chem., 274(12):8282-90, 1999.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Laughlin et al., J. Virol., 60:515-524, 1986.

Lee et al., Nature, 294:228, 1981.

Lee et al., Proc. Soc. Exp. Biol. Med., 216(3):319-57, 1997.

Le Gal La Salle et al., Science, 259:988-990, 1993.

Lebkowski et al., Mol. Cell. Biol., 8:3988-3996, 1988.

Lehnherr and Yarmolinski, Proc. Natl. Acad. Sci. USA, 92(8):3274-7, 1995.

Levenson et al., Hum. Gene Ther., 9(8):1233-6, 1998.

Levinson et al., Nature, 295:79, 1982.

Levrero et al., Gene, 101: 195-202, 1991.

Lin et al., Mol. Cell. Biol., 10:850, 1990.

Luo et al., Blood, 82(Supp.):1,303A, 1994.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Macejak and Sarnow, Nature, 353(6339):90-94, 1991.

Majors and Varmus, Proc. Nat'l Acad. Sci. USA., 80:5866, 1983.

Mann et al., Cell, 33:153-159, 1983.

Mann et al., J. Appl. Physiol. 61:1667-1676, 1986.

Manz et al., J. Chromatogr., 593:253-258, 1992.

McCarty et al., J. Virol., 65:2936-2945, 1991.

McLaughlin et al., J. Virol., 62:1963-1973, 1988.

McNeall et al., Gene, 76:81, 1989.

Mewes et al., Nature (Supplement), 387:7-8, 1997.

Miksicek et al., Cell, 46:203, 1986.

Mordacq and Linzer, Genes and Dev., 3:760, 1989.

Moreau et al., Nucl. Acids Res., 9:6047, 1981.

Muesing et al., Cell, 48:691, 1987.

Muzyczka, Curr. Top. Microbiol. Immunol., 158:97-129, 1992.

Ng et al., Nuc. Acids Res., 17:601, 1989.

Nicolas and Rubinstein, In: Vectors: A survey of molecular cloning vectors and their uses, Rodriguez and Denhardt (eds.), Stoneham: Butterworth, 494-513, 1988.

Nicolau and Sene, Biochim. Biophys. Acta, 721:185-190, 1982

Nicolau et al., Methods Enzymol., 149:157-176, 1987.

Nomoto et al., Gene, 236(2):259-71, 1999.

Ohara et al., Proc. Natl. Acad. Sci. USA, 86(15):5673-5677, 1989.

Ohi et al., Gene, 89L:279-282, 1990.

Ondek et al., EMBO J., 6:1017, 1987.

Ornitz et al., Mol. Cell. Biol., 7:3466, 1987.

Palmiter et al., Nature, 300:611, 1982.

Paskind et al., Virology, 67:242-248, 1975.

Pech et al., Mol. Cell. Biol., 9:396, 1989.

Pelicic, Reyrat, Gicquel, J. Bacteriol., 178(4):1197-9, 1996.

Pelletier and Sonenberg., Nature, 334(6180):320-325, 1988.

Perales et al., Proc. Natl. Acad. Sci. USA, 91:4086-4090, 1994.

Perez-Stable and Constantini, Mol. Cell. Biol., 10:1116, 1990.

Picard and Schaffner, Nature, 307:83, 1984.

Pinkert et al., Genes and Dev., 1:268, 1987.

Ponta et al., Proc. Nat'l Acad. Sci. USA., 82:1020, 1985.

Porton et al., Mol. Cell. Biol., 10: 1076, 1990.

Potter et al., Proc. Natl. Acad. Sci. USA, 81:7161-7165, 1984.

Prasher, Trends in Biochem. Sci., 11:320-323, 1995.

Queen and Baltimore, Cell, 35:741, 1983.

Quinn et al., Mol. Cell. Biol., 9:4713, 1989.

Racher et al., Biotechnology Techniques, 9:169-174, 1995.

Ragot et al., Nature, 361:647-650, 1993.

Recorbet et al., Appl. Environ. Microbiol., 59(5):1361-6, 1993.

Redondo et al., Science, 247:1225, 1990.

Reisman and Rotter, Mol. Cell. Biol., 9:3571, 1989.

Resendez Jr. et al., Mol. Cell. Biol., 8:4579, 1988.

Rich et al., Hum. Gene Ther., 4:461-476, 1993.

Ridgeway, In: Vectors: A survey of molecular cloning vectors and their uses. Rodriguez and Denhardt, eds., Stoneham: Butterworth, 467-492, 1988.

Ripe et al., Mol. Cell. Biol., 9:2224, 1989.

Rittling et al., Nucl. Acids Res., 17:1619, 1989.

Rosen et al., Cell, 41:813, 1988.

Rosenfeld et al., Cell, 68:143-155, 1992.

Rosenfeld et al., Science, 252:431-434, 1991.

Roux et al., Proc. Natl. Acad. Sci. USA, 86:9079-9083, 1989.

Ruiz-Echevarria et al., J. Mol. Biol., 247(4):568-77, 1995.

Ruther et al., Proc. Natl. Acad. Sci. USA, 79:6852-6855, 1982.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Samulski et al., EMBO J., 10:3941-3950, 1991.

Samulski et al., J. Virol., 63:3822-3828, 1989.

Santos-Sierra et al., FEMS Micro. Let., 152:51-56, 1997.

Satake et al., J Virology, 62:970, 1988.

Schaffner et al., J. Mol. Biol., 201:81, 1988.

Searle et al., Mol. Cell. Biol., 5:1480, 1985.

Sharp and Marciniak, Cell, 59:229, 1989.

Shaul and Ben-Levy, EMBO J., 6:1913, 1987.

Shelling and Smith, Gene Therapy. 1: 165-169, 1994.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stenger et al., Science, 282(5386):121-5, 1998.

Stephens and Hentschel, Biochem. J., 248:1, 1987.

Stills, The Biology of the Laboratory Rabbit, eds. P. J. Manning et al., Academic Press, Inc., New York, pp. 435-448, 1994.

Stratford-Perricaudet and Perricaudet., In: Human Gene Transfer, Eds, Cohen-Haguenauer and Boiron, Editions John Libbey Eurotext, France, 51-61, 1991.

Stratford-Perricaudet et al., Hum. Gene. Ther., 1:241-256, 1991.

Stuart et al., Nature, 317:828, 1985.

Studier et al., Methods Enzymol. 185:60-89, 1990.

Sullivan and Peterlin, Mol. Cell. Biol., 7:3315, 1987.

Sutcliffe et al., Science, 219:660-666, 1984.

Swartzendruber and Lehman, J. Cell. Physiology, 85:179, 1975.

Sykes and Johnston, DNA and Cell Biol., 18:521-531, 1999.

Tabor et al., Proc. Natl. Acad. Sci. USA, 84:4767, 1987.

Takebe et al., Mol. Cell. Biol., 8:466, 1988.

Tang, et al., Nature 356:152-154, 1992.

Tavernier et al., Nature, 301:634, 1983.

Taylor and Kingston, Mol. Cell. Biol., 10: 165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati, ed., New York: Plenum Press, 149-188, 1986.

Thiesen et al., J Virology, 62:614, 1988.

Top et al., J. Infect. Dis., 124:155-160, 1971.

Tratschin et al., Mol. Cell. Biol., 4:2072-2081, 1984.

Tratschin et al., Mol. Cell. Biol., 5:32581-3260, 1985.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Trudel et al., Biotechniques, 20(4):684-93, 1996.

Tsien, Annu. Rev. Biochem., 67:509-544, 1998.

Tsumaki et al., J. Biol. Chem., 273(36):22861-4, 1998.

Tur-Kaspa et al., Mol. Cell Biol., 6:716-718, 1986.

Tyndall et al., Nuc. Acids. Res., 9:6231, 1981.

Ulmer and Liu, Trends Microbiol., 4(5):169-70, 1996.

Vannice and Levinson, J. Virology, 62:1305, 1988.

Vasseur et al., Proc. Nat'l. Acad. Sci. USA., 77:1068, 1980.

Wagner et al., Science, 260:1510-1513, 1990.

Walker et al., Proc. Natl. Acad. Sci. USA, 89(1):392-396, 1992.

Walsh et al., J. Clin. Invest., 94:1440-1448, 1994.

Wang and Calame, Cell, 47:241, 1986.

Weber et al., Cell, 36:983, 1984.

Wei et al., Gene Therapy, 1:261-268, 1994.

Weinberger et al. Mol. Cell. Biol., 8:988, 1984.

Weinberger et al., Science, 228:740-742, 1985.

Weinstock, Methods Enzymol., 154:156-163, 1987.

Weinstock et al., Proc. Natl. Acad. Sci. USA, 80:4432-4436, 1983.

Winoto and Baltimore, Cell, 59:649, 1989.

Wolf et al., Comput. Appl. Biosci., 4(1):187-191, 1988.

Wong et al., Gene, 10:87-94, 1980.

Woolley and Mathies, Proc. Natl. Acad. Sci. USA, 91:11348-11352, 1994.

Wu and Wu, Adv. Drug Delivery Rev., 12:159-167, 1993.

Wu et al., Genomics, 4(4):560-9, 1989.

Wu and Wu, J. Biol. Chem., 262:4429-4432, 1987.

Yang et al., J. Virol., 68:4847-4856, 1994.

Yang et al., Proc. Nat'l Acad. Sci. USA, 87:9568-9572, 1990.

Yazynin et al., FEBS Lett., 452(3):351-4, 1999.

Yoder et al., Blood, 82 (Supp.):1:347A, 1994.

Young, Microbiol. Rev., 56(3):430-81, 1992.

Yutzey et al. Mol. Cell. Biol., 9:1397, 1989.

Zhao-Emonet et al., Biochim. Biophys. Acta., 1442(2-3):109-19, 1998.

Zhou et al., Exp. Hematol. (NY), 21:928-933, 1993.

Zhou, et al., J. Exp. Med., 179:1867-1875, 1994.

What is claimed is:

1. An ORF selection vector comprising:
(a) a promoter;
(b) a start codon operably linked to the promoter;
(c) a reporter gene that is positioned downstream from both the promoter and the start codon and is out of frame.

2. The ORF selection vector of claim 1, wherein a nucleic acid sequence is inserted between the start codon and the reporter gene such that the reporter gene is in frame.

3. The ORF selection vector of claim 2, wherein the inserted nucleic acid sequence is genomic DNA.

4. The ORF selection vector of claim 3, wherein the genomic DNA is from a eukaryote.

5. The ORF selection vector of claim 3, wherein the genomic DNA is from a prokaryote.

6. The ORF selection vector of claim 2, wherein the genomic DNA is from a pathogen.

7. The ORF selection vector of claim 6, wherein the genomic DNA is from a parasite.

8. The ORF selection vector of claim 7, wherein the parasite is Plasmodium falciparum.

9. The ORF selection vector of claim 7, wherein the parasite is Neospora caninum.

10. The ORF selection vector of claim 7, wherein the parasite is Trypanosoma cruzi.

11. The ORF selection vector of claim 1, wherein the reporter gene lacks a start codon.

12. The ORF selection vector of claim 1, wherein the reporter gene encodes a gene product that is nonenzymatic.

13. The ORF selection vector of claim 12, wherein the gene product is GFP.

14. The ORF selection vector of claim 1, wherein the reporter gene is a death gene.

15. The ORF selection vector of claim 14, wherein the death gene encodes an enzyme, a DNA replication inhibitor, or a membrane disruptor.

16. The ORF selection vector of claim 15, wherein the enzyme is barnase, colicin, or SacB.

17. The ORF selection vector of claim 15, wherein the DNA replication inhibitor is CcdB, Kid, or GATA.

18. The ORF selection vector of claim 15, wherein the membrane disruptor is Hok, holins, or granulysin.

19. The ORF selection vector of claim 14, wherein the death gene encodes Doc.

20. The ORF selection vector of claim 2, wherein the nucleic acid sequence is part or all of an ORF of a gene.

21. The ORF selection vector of claim 1, wherein the promoter is a T7 promoter.

22. The ORF selection vector of claim 1, further comprising a restriction endonuclease site between the start codon and the reporter gene.

23. The ORF selection vector of claim 1, further comprising an origin of replication.

24. The ORF selection vector of claim 1, further comprising a selectable marker.

25. The ORF selection vector of claim 24, wherein the selectable marker is in frame and expressed in a host cell.

26. A method of producing an ORF selection vector comprising:
(a) contacting genomic DNA with a restriction endonuclease;
(b) obtaining an ORF selection vector comprising:
(i) a promoter;
(ii) a start codon, wherein the start codon operably linked to the promoter;
(iii) a reporter gene that is positioned downstream from both the promoter and the start codon and is out of frame;
(c) contacting the ORF selection vector with a restriction endonuclease; and
(d) ligating a genomic restriction endonuclease DNA fragment generated from step (a) with the linearized ORF selection vector.

27. The method of claim 26, further comprising transfecting a host cell with the ligated ORF selection vector.

28. The method of claim 27, wherein the host cell is a bacterial host cell.

29. The method of claim 26, wherein the ligated ORF selection vector is capable of expressing the reporter gene.

30. The method of claim 26, wherein the genomic restriction endonuclease DNA fragment comprises a portion of an ORF.

31. The method of claim 30, wherein the DNA fragment is from a eukaryote.

32. The method of claim 30, wherein the DNA fragment is from a prokaryote.

33. The method of claim 30, wherein the DNA fragment is from a pathogen.

34. The method of claim 30, wherein the DNA fragment is from a parasite.

35. The method of claim 34, wherein the parasite is Plasmodium falciparum.

36. The method of claim 34, wherein the parasite is Neospora caninum.

37. The method of claim 34, wherein the parasite is Trypanosoma cruzi.

38. The method of claim 26, wherein the reporter gene lacks a start codon.

39. The method of claim 26, wherein the reporter gene encodes a gene product that is nonenzymatic.

40. The method of claim 39, wherein the gene product is GFP.

41. The method of claim 26, wherein the reporter gene is a death gene.

42. The method of claim 41, wherein the death gene encodes an enzyme, a DNA replication inhibitor, or a membrane disruptor.

43. The method of claim 42, wherein the enzyme is barnase, colicin, or SacB.

44. The method of claim 42, wherein the DNA replication inhibitor is CcdB, Kid, or GATA.

45. The method of claim 42, wherein the membrane disruptor is Hok, holins, or granulysin.

46. The method of claim 41, wherein the death gene encodes Doc.

47. The method of claim 26, wherein the promoter of the ORF selection vector is a T7 promoter.

48. The method of claim 26, wherein the restriction endonuclease contacted with the genomic DNA creates a site compatible with the site created by the restriction endonuclease contacted with the ORF selection vector.

49. The method of claim of claim 26, further comprising contacting the ORF selection vector with a phosphatase after it is contacted with a restriction endonuclease.

50. A method of identifying at least a portion of an ORF comprising:
(a) contacting genomic DNA with a restriction endonuclease;
(b) obtaining an ORF selection vector comprising:
(i) a promoter;
(ii) a start codon operably linked to the promoter;
(iii) a reporter gene that is positioned downstream from both the promoter and the start codon and is out of frame;
(c) contacting the ORF selection vector with a restriction endonuclease;
(d) ligating a genomic restriction endonuclease DNA fragment generated from step (a) with the linearized ORF selection vector;
(e) transfecting a host cell with the ligated selection vector;
(f) determining whether the reporter gene is expressed.

51. The method of claim 50, wherein the genomic restriction endonuclease DNA fragment comprises a portion of an ORF.

52. The method of claim 51, wherein the DNA fragment is from a eukaryote.

53. The method of claim 51, wherein the DNA fragment is from a prokaryote.

54. The method of claim 51, wherein the DNA fragment is from a pathogen.

55. The method of claim 51, wherein the DNA fragment is from a parasite.

56. The ORF selection vector of claim 55, wherein the parasite is Plasmodium falciparum.

57. The ORF selection vector of claim 55, wherein the parasite is Neospora caninum.

58. The ORF selection vector of claim 55, wherein the parasite is Trypanosoma cruzi.

59. The method of claim 50, wherein the reporter gene lacks a start codon.

60. The method of claim 50, wherein the reporter gene is nonselectable.

61. The method of claim 60, wherein the reporter gene encodes a gene product that is nonenzymatic.

62. The method of claim 61, wherein the gene product is GFP.

63. The method of claim 50, wherein the reporter gene is a death gene.

64. The method of claim 63, wherein the death gene encodes an enzyme, a DNA replication inhibitor, or a membrane disrupter.

65. The method of claim 64, wherein the enzyme is barnase, colicin, or SacB.

66. The method of claim 64, wherein the DNA replication inhibitor is CcdB, Kid, or GATA.

67. The method of claim 64, wherein the membrane disruptor is Hok, holins, or granulysin.

68. The method of claim 63, wherein the death gene encodes Doc.

69. The method of claim 50, wherein the promoter of the ORF selection vector is a T7 promoter.

70. A method of inducing an immune response in an animal comprising:
(a) obtaining an ORF selection vector comprising:
(i) a promoter;
(ii) a start codon operably linked to the promoter;
(iii) a reporter gene that is positioned downstream from both the promoter and the start codon;
(iv) at least a part of a genomic ORF that is positioned between the start codon and the reporter gene;
(b) identifying an ORF by determining whether the reporter gene is expressed;
(c) if the reporter gene is expressed, subcloning the ORF into an expression construct lacking the reporter gene;
(d) introducing the expression construct into an the animal in a manner effective to induce an immune response against one or more antigens that may be encoded by the construct.

71. The method of claim 70, wherein the ORF is from a eukaryote.

72. The method of claim 71, where in the ORF is from a tumor cell.

73. The method of claim 70, wherein the ORF is from a prokaryote.

74. The method of claim 70, wherein the DNA fragment is from a pathogen.

75. The method of claim 70, wherein the DNA fragment is from a parasite.

76. The method of claim 75, wherein the parasite is Plasmodium falciparum.

77. The method of claim 75, wherein the parasite is Neospora caninum.

78. The method of claim 75, wherein the parasite is Trypanosoma cruzi.

79. The method of claim 70, wherein the reporter gene lacks a start codon.

80. The method of claim 70, wherein the reporter gene encodes a gene product that is nonenzymatic.

81. The method of claim 80, wherein the gene product is GFP.

82. The method of claim 70, wherein the reporter gene is toxic to a host cell.

83. The method of claim 70, wherein the promoter of the ORF selection vector is a T7 promoter.

84. The method of claim 70, wherein the expression construct contains a eukaryotic promoter.

85. The method of claim 84, wherein the eukaryotic promoter is from the same species as the animal.

86. The method of claim 70, further comprising testing the animal for an immune response.

87. The method of claim 86, wherein the testing comprises challenging the animal with an expression product of the ORF.

88. The method of claim 70, further comprising obtaining antibodies generated in response to one or more antigens encoded by the introduced second construct.

89. A method of preparing an antigen comprising:
(a) obtaining an ORF selection vector comprising:
(i) a promoter;
(ii) a start codon operably linked to the promoter;
(iii) a reporter gene that is positioned downstream from both the promoter and the start codon;
(iv) at least a part of a genomic ORF that is positioned between the start codon and the reporter gene;
(b) identifying an ORF by determining whether the reporter gene is expressed;
(c) if the reporter gene is expressed, subcloning the ORF into an expression construct lacking the reporter gene;
(d) administering to an animal a pharmaceutical composition comprising one or more expression constructs; and
(e) identifying the antigen or antigens so expressed.

90. A kit for identifying an antigen comprising:
(a) an ORF selection vector comprising:
(i) a promoter;
(ii) a start codon operably linked to the promoter;
(iii) a reporter gene that is positioned downstream from both the promoter and the start codon and is out of frame; and
(iv) a restriction endonuclease site between the reporter gene and the start codon;
(b) an expression construct lacking the reporter gene.


SEQUENCE LISTING (3 sequences)

SEQ ID NO: 1 - DNA, 160 bp, Artificial Sequence
  Description of Artificial Sequence  Synthetic Primer
ggtgcagatcttggatctcgtcccgcgaaattaatacgactcactatagggagacccaacggtttccctctagaaataattttgtttcacttcaagaaggagatatacatatgggatccgggcaggtaagtatcaaggttacaagacaagcttacatatg

SEQ ID NO: 2 - DNA, 167 bp, Artificial Sequence
  Description of Artificial Sequence  Synthetic Primer
ggtgcagatcttggatctcgtcccgcgaaattaatacgactcactatagggagaccacaacggtttccctctagaaataattttgtttcacttcaagaaggagatatacatatgggatcattaattaacggatccgggcgcgccgctgcagctcaagcttacatgcg

SEQ ID NO: 3 - DNA, 167 bp, Artificial Sequence
  Description of Artificial Sequence  Synthetic Primer
ggtgcagatcttggatctcgtcccgcgaaattaatacgactcactatagggagaccacaacggtttccctctagaaataattttgtttcacttcaagaaggagatatacatatgggatcattaattaacggcgccgggcgcgccgctgcagctcaagcttacatgcg